(19) United States
(12) Patent Application Publication
(10) Pub. No.: US 2026/0242786 A1
(43) Pub. Date: Aug. 20, 2026

(54) HETERODUPLEX RNA EDITING OLIGONUCLEOTIDE COMPLEXES

(71) Applicant: ProQR Therapeutics II B.V., Leiden (NL)

(72) Inventor: Peter Christian DE VISSER, Leiden (NL)

(73) Assignee: ProQR Therapeutics II B.V., Leiden (NL)

(21) Appl. No.: 19/122,555

(22) PCT Filed: Oct. 20, 2023
(86) PCT No.: PCT/EP2023/079290

§ 371 Date: Apr. 18, 2025

(30) Foreign Application Priority Data
Oct. 21, 2022 (GB) 2215614.5

(51) Int. Cl. C12N 15/113; A61K 31/7088; C12N 9/78

(52) U.S. Cl. CPC C12N 15/113; A61K 31/7088; C12N 9/78; C12Y 305/04004; C12N 2310/11; C12N 2310/3125; C12N 2310/315; C12N 2310/3515; C12N 2310/3519

(57) ABSTRACT

The invention relates to a heteroduplex RNA editing oligonucleotide complex (HEON) comprising a first nucleic acid strand annealed to a second nucleic acid strand, for use in the targeted deamination of a target adenosine present in a target RNA molecule, wherein the first nucleic acid strand is capable of hybridizing to a stretch within the target RNA molecule that includes the target adenosine, and thereby forms a double-stranded nucleic acid complex that can recruit an enzyme with deaminase activity to deaminate the target adenosine to an inosine. The first nucleic acid strand and/or the second nucleic acid strand in the HEON is bound to a hydrophobic moiety, and optionally to a cell-targeting ligand.

### TECHNICAL FIELD

The invention relates to the field of medicine, especially to the field of RNA editing, whereby an RNA molecule in a cell is targeted by an antisense oligonucleotide to specifically change a target nucleotide present in the target RNA molecule. The invention relates to double stranded oligonucleotide complexes wherein a first oligonucleotide, that is designed to hybridize to a specific (pre-) mRNA target sequence in the cell, is complexed with a second oligonucleotide for improved stability, cellular delivery, and intracellular trafficking, and through this results in increased and/or more efficient RNA editing.

### BACKGROUND

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result, exons may be included or skipped. The enzymes catalysing adenosine deamination are within an enzyme family of ADARs, which include human deaminases hADAR1 and hADAR2, as well as hADAR3. However, for hADAR3 no deaminase activity has been demonstrated.

The use of oligonucleotides to edit a target RNA applying adenosine deaminase has been described (e.g., Woolf et al. 1995. PNAS 92:8298-8302; Montiel-Gonzalez et al. PNAS 2013, 110 (45): 18285-18290; Vogel et al. 2014. Angewandte Chemie Int Ed 53:267-271). A disadvantage of the method described by Montiel-Gonzalez et al. (2013) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995) that were 100% complementary to the target RNA sequences suffered from severe lack of specificity: nearly all adenosines in the target RNA strand that was complementary to the antisense oligonucleotide were edited.

It is known that ADAR may act on any dsRNA. Through a process sometimes referred to as ‘promiscuous editing’, the enzyme will edit multiple A's in the dsRNA. Hence, there is a need for methods and means that circumvent such promiscuous editing and only target specific adenosines in a target RNA molecule to become therapeutic applicable. Vogel et al. (2014) showed that such off-target editing can be suppressed by using 2′-O-Me-modified nucleosides in the oligonucleotide at positions opposite to adenosines that should not be edited and used a non-modified nucleoside directly opposite to the specifically targeted adenosine on the target RNA. However, the specific editing effect at the target nucleotide has not been shown to take place without the use of recombinant ADAR enzymes having covalent bonds with the AON. Several publications have now shown that the recruitment of endogenous ADAR (hence without the need for an exogenous and/or recombinant source) is feasible while maintaining a specificity in which a single adenosine within a target RNA molecule can be targeted and deaminated to an inosine. WO2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop/hairpin structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion, there is no need for conjugated entities or presence of modified recombinant ADAR enzymes. WO2016/097212 describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the AON itself, and are thought to attract (endogenous) ADAR. Similar stem-loop structure-comprising systems for RNA editing have been described in WO2017/050306, WO2020/001793, WO2017/010556, WO2020/246560, and WO2022/078995.

WO2017/220751 and WO2018/041973 describe a next generation type of AONs that do not comprise such a stem-loop structure but that are (almost fully) complementary to the targeted area except for one or more mismatching nucleotides, wobbles, or bulges. The sole mismatch may be at the site of the nucleoside opposite the target adenosine, but in other embodiments AONs were described with multiple bulges and/or wobbles when attached to the target sequence area. It appeared possible to achieve in vitro, ex vivo and in vivo RNA editing with AONs lacking a stem-loop structure and with endogenous ADAR enzymes when the sequence of the AON was carefully selected such that it could attract/recruit ADAR. The ‘orphan nucleoside’, which is defined as the nucleoside in the AON that is positioned directly opposite the target adenosine in the target RNA molecule, did not carry a 2′-O-Me modification. The orphan nucleoside can be a deoxyribonucleoside (DNA), wherein the remainder of the AON did carry 2′-O-alkyl modifications at the sugar entity (such as 2′-O-Me), or the nucleotides directly surrounding the orphan nucleoside contained particular chemical modifications (such as DNA in comparison to RNA) that further improved the RNA editing efficiency and/or increased the resistance against nucleases. Such effects could even be further improved by using sense oligonucleotides (SONs) that ‘protected’ the AONs against breakdown (described in WO2018/134301). The use of chemical modifications and particular structures in oligonucleotides that could be used in ADAR-mediated editing of specific adenosines in a target RNA have been the subject of numerous publications in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in WO2011/005761, WO2014/010250, WO2014/012081, WO2015/107425, WO2017/015575 (HTT), WO2017/062862, (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2017/160741, WO2017/192664, WO2017/192679 WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, and WO2022/099159. Next to these disclosures, an extensive number of publications relate to the targeting of specific RNA target molecules, or specific adenosines within such RNA target molecules, be it to repair a mutation that resulted in a premature stop codon, or other mutation causing disease. Examples of such disclosures in which adenosines are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1).

Despite the numerous and wide variety of achievements outlined above, there remains a need for improved compounds that can utilise (endogenous) cellular pathways and enzymes that have deaminase activity, such as naturally expressed ADAR enzymes to edit specifically and more efficiently endogenous nucleic acids in mammalian cells, even in whole organisms, to alleviate disease.

### SUMMARY OF THE INVENTION

The invention relates to a heteroduplex RNA editing oligonucleotide complex (HEON) for use in the deamination of a target adenosine present in a target RNA molecule in a cell, wherein the HEON comprises a first nucleic acid strand that is annealed to a (partly or fully) complementary second nucleic acid strand, wherein the first nucleic acid strand is complementary to a stretch of (preferably at least 8) nucleotides in the target RNA molecule that includes the target adenosine, wherein the first nucleic acid strand when it is hybridized to the target RNA molecule can recruit an enzyme with deaminase activity present in the cell allowing for the deamination of the target adenosine, and wherein the first and/or second nucleic acid strand is bound to a hydrophobic moiety. In one embodiment, the hydrophobic moiety is a lipid, a hydrophobic vitamin, or a steroid. In one embodiment, the hydrophobic vitamin is vitamin E or analog thereof. In one embodiment, the steroid is cholesterol or analog thereof. In a preferred embodiment, the invention relates to a HEON wherein the hydrophobic moiety is bound to the 5′ terminus, to the 3′ terminus, and/or to an internal position of the first nucleic acid strand and/or of the second nucleic acid strand. Preferably, it is bound to the 5′ terminus of the second nucleic acid strand. In another embodiment, the hydrophobic moiety is bound to the 3′ terminus of the second nucleic acid strand. In one embodiment, the hydrophobic moiety is bound via a cleavable linker. In one embodiment, the invention relates to a HEON according to the invention, wherein the HEON is further bound to a ligand that enables cell-specific targeting. A preferred ligand is a N-acetylgalactosamine (GalNAc) moiety or cluster that allows more efficient entry into liver cells (such as hepatocytes) through the interaction with asialoglycoprotein receptors.

The HEON according to the invention comprises a first nucleic acid strand that, after entry into the cell, attaches to the target RNA molecule and forms a double-stranded nucleic acid complex with the endogenous target (pre-) mRNA and thereby can attract (recruit) an enzyme with deaminase activity. Preferably, the enzyme with deaminase activity is endogenously present in the cell in which the first nucleic acid strand performs its action, and preferably the enzyme is an endogenous ADAR enzyme, preferably a human ADAR enzyme, more preferably human ADAR1 or ADAR2.

The invention also relates to a pharmaceutical composition comprising a HEON according to the invention, further comprising a pharmaceutically acceptable carrier and/or solvent.

The invention also relates to a HEON according to the invention, or a pharmaceutical composition according to the invention, for use in the treatment or prevention of a disorder selected from the group consisting of: Hurler Syndrome, alpha-1-antitrypsin (A1AT) deficiency, (familial) hypercholesterolemia, Parkinson's disease, Rett syndrome, Stargardt Disease, Citrullinemia Type 1, autosomal recessive non-syndromic hearing loss, X-linked retinoschisis, argininosuccinate lyase deficiency, Duchenne/Becker muscular dystrophy, Non-Alcoholic Steatohepatitis (NASH), Myotonic dystrophy type I, Myotonic dystrophy type II, Huntington's disease, Usher syndrome (such as Usher syndrome type I, II, and III), Charcot-Marie-Tooth disease, Cystic fibrosis, Alzheimer's disease, albinism, Amyotrophic lateral sclerosis, Asthma, β-thalassemia, Epileptic Encephalopathy, CADASIL syndrome, Chronic Obstructive Pulmonary Disease (COPD), Distal Spinal Muscular Atrophy (DSMA), Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase deficiency, Haemophilia, Hereditary Hemochromatosis, Hereditary Cancer predisposing Syndrome, Hunter Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anaemia, Spinal Muscular Atrophy, Tay-Sachs Disease, X-linked immunodeficiency, Sturge-Weber Syndrome, and cancer, such as breast and lung cancer. Even though many disorders may be caused by a specific G to A mutation, which may result in a premature termination codon, and which may be reversed by using a HEON according to the invention, many disorders may also be the result of aberrant splicing events, loss-of-function or gain-of-function mutations, aberrant phosphorylation events, internal enzymatic cleavage events, etc, and may be treatable using a HEON according to the present invention.

In one embodiment, the invention relates to a method for the deamination of at least one target adenosine in a target RNA in a cell, the method comprising the steps of: (i) providing the cell with a HEON as defined herein; (ii) allowing annealing of the first nucleic acid strand to the target RNA in the cell to form a double stranded nucleic acid molecule; (iii) allowing an ADAR enzyme to complex with the double stranded nucleic acid molecule between the guide oligonucleotide and the target RNA and to deaminate the target adenosine in the target RNA to an inosine; and (iv) optionally identifying the presence of the deaminated nucleotide in the target RNA.

### BRIEF DESCRIPTION OF THE DRAWINGS

One or more embodiments of the invention will now be described, by way of example only, with reference to the accompanying drawings, in which:

FIG. 1 (A) shows several examples of HEONs. Open circles represent nucleotides of the first nucleic acid strand and dark circles represent nucleotides of the second nucleic acid strand. The upper strand is shown from 5′ to 3′, while then the lower strand is 3′ to 5′. The first nucleic acid strand and the second nucleic acid strand are not connected to each other, or linked through a linker, a hairpin, or stem-loop structure. They anneal to each other via Watson/Crick base pairing. The first and second nucleic acid strand may have an equal length but do not necessarily have to overlap 100%. The first and second strands may have different lengths and the complementarity of the smaller nucleic acid strand may be complete or partial to the other strand. The lengths of the nucleic acid strands shown here is by no means limiting to the lengths of the first nucleic acid strand and second nucleic acid strand of the HEONs of the present invention and lengths are purely depicted as examples. All shown complexes are HEONs, in which the upper complex is a fully complementary HEON, whereas the other complexes are partially complementary HEONs (pHEONs), because not each nucleotide in one strand has a partnering complementary nucleotide in the other strand. However, if the first nucleic acid strand and the second nucleic acid strand can anneal and form a heteroduplex oligonucleotide complex, it is part of the present invention, if it is bound to a hydrophobic moiety as disclosed herein. Any nucleotide in the open circles may be the orphan nucleotide of the first nucleic acid strand, although it is preferred that there are at least four nucleotides 5′ and 3′ from the orphan nucleotide within the first nucleic acid strand. The HEONs as shown may be fully or partially complementary, and there may be mismatches between both nucleic acid strands. There may also be internal loops in the second nucleic acid strand as shown at the bottom, in which the loop may be a linker or may be formed by nucleotides that are not annealing to the first nucleic acid strand. Such a linker may also be present in any of the other pHEONs shown. (B) shows the potential position of the hydrophobic moiety that is part of the HEON according to the present invention. It is here shown for the 100% fully complementary HEON but is valid for all possible HEONs according to the invention, such as those exemplified in (A). The hydrophobic moiety may be bound to the 5′ and/or the 3′ terminal nucleotide of the first nucleic acid strand, but it may also be bound to the 5′ and/or 3′ terminal nucleotide of the second nucleic acid strand, and it may also be bound to an internal nucleotide in the first and/or second nucleic acid strand. (C) shows an example of a HEON according to the present invention, in which a hydrophobic moiety is bound to the 5′ terminus of the second nucleic acid strand (as shown in (B)) and a ligand (e.g., a GalNAc moiety, a linear GalNAc moiety, or tri-antennary GalNAc cluster; here represented by diamond shaped icons) is bound to the 5′ terminus of the first nucleic acid strand. As outlined herein, variations of these positions are possible (e.g., the hydrophobic moiety at the 3′ terminus of a nucleic acid strand, or at an internal nucleotide, and the ligand to the 3′ terminus of the other nucleic acid strand, etc.).

FIG. 2 (A) shows part of the mouse Amyloid Beta Precursor Protein (mApp) target RNA sequence (SEQ ID NO: 1) from 5′ to 3′ with the target adenosine underlined and in bold. (B) shows the sequence of the oligonucleotides used to generate heteroduplex RNA editing complexes in which the 25 nt mEON1 (RM3835; SEQ ID NO:2) represents the first nucleic acid strand (the RNA editing producing oligonucleotide, which is antisense to the target mAPP sequence) and in which rEON1 (RM4223; SEQ ID NO:3) and rEON2 (RM4224; SEQ ID NO:4) represent second nucleic acid strands that are complementary to mEON1 (fully and partly, respectively). Toc-rEON1 (RM4225) and Toc-rEON2 (RM4226) have the same sequence, respectively, but are bound to tocopherol at the 5′ end. Chol-rEON1 (RM4227) and Chol-rEON2 (RM4228) have the same sequence, respectively, but are bound to cholesterol at the 5′ end. RM4237 (SEQ ID NO:5), RM4238 (SEQ ID NO:6), and RM4239 (SEQ ID NO:7) are second nucleic acid strands that are—like rEON2—complementary to a part of mEON1 and are shortened at their 3′ ends, and all bound to cholesterol at the 5′ end. The positions of the mEON1 first nucleic acid strand oligonucleotide are incrementing positively (+) towards the 5′ terminus, and negatively (−) incremented towards the 3′ terminus, wherein the orphan nucleotide (the orphan cytidine) is at position 0. dZ represents the orphan nucleotide, here a deoxyribonucleoside (upper case, bold) carrying a Benner's base (also referred to as 6-amino-5-nitro-2 (1H)-pyridone; Yang et al. Nucl Acid Res 2006. 34(21):6095-6101). A, G, C, and T nucleosides (upper case, bold) are DNA. Underlined A nucleoside (not bold) is 2′-MOE modified. C nucleoside (upper case, bold) is 5-methyl-C DNA. Underlined C nucleoside (not bold) is 2′-MOE modified 5-methyl-C. c, a, u, and g nucleosides (lower case, not bold, not underlined) are 2′-OMe modified. Gf, Uf, Af, and Cf (upper case, not bold) are nucleosides in which the ribose comprises a 2′-fluoro substitution. Asterisks depict phosphorothioate (PS) linkages. The “\hat{ }” symbol refers to a methylphosphonate (MP) linkage. The “!” symbol refers to a PNdmi linkage. The triplet with the orphan nucleotide (here dZ) in the middle and its two surrounding nucleotides is given by a grey box. (C) shows the percentage editing as determined after dPCR using the indicated oligonucleotides and heteroduplex editing oligonucleotides (HEONs) after IVT injection, wherein the results of the retina from the left and right eye were averaged.

FIG. 3 (A) shows the percentage editing on a mApp target transcript in vivo, in the liver of mice, as determined after dPCR using the indicated oligonucleotides (identical to what has been shown in FIG. 2) and HEONs after IV injection. (B) shows the percentage editing on a mApp target transcript in vivo, in the kidneys of mice, as determined after dPCR using the indicated oligonucleotides (identical to what has been shown in FIG. 2) and HEONs after IV injection. (C) shows the percentage editing on a mApp target transcript in vivo, in the lungs of mice, as determined after dPCR using the indicated oligonucleotides (identical to what has been shown in FIG. 2) and HEONs after IV injection. (D) shows the percentage editing on a mApp target transcript in vivo, in the spleen of mice, as determined after dPCR using the indicated oligonucleotides (identical to what has been shown in FIG. 2) and HEONs after IV injection. (E) shows the percentage editing on a mApp target transcript in vivo, in the heart muscle of mice, as determined after dPCR using the indicated oligonucleotides (identical to what has been shown in FIG. 2) and HEONs after IV injection.

FIG. 4 shows the percentage editing that is observed in mouse retinal pigment epithelial (RPE) cells after gymnotic uptake of mEON1 (RM3835) alone, or after gymnotic uptake of a HEON comprising mEON1 (RM3835) as a first nucleic acid strand and RM4238 (see FIG. 2B) as a second nucleic acid strand, and after gymnotic uptake of a HEON comprising mEON1 (RM3835) as a first nucleic acid strand and RM4239 (see FIG. 2B) as a second nucleic acid strand, clearly showing the beneficial properties of a HEON in which the second nucleic acid strand is bound to cholesterol.

### DETAILED DESCRIPTION

### Overall Background

The double-stranded nucleic acid complex of the present invention comprises a first nucleic acid strand and a second nucleic acid strand. In a double-stranded nucleic acid complex of the present invention, the second nucleic acid strand is annealed to the first nucleic acid strand via hydrogen bonds of complementary base pairs. When the first nucleic acid strand and the second nucleic acid strand are annealed to each other, the double-stranded complex is often herein and elsewhere referred to as “heteroduplex RNA editing oligonucleotide” complexes, as “HEON” complexes, or simply as “HEONs” in short. The first and second nucleic acid strand are not linked to each other via a linker or through a stem-loop structure. Except that the first nucleic acid strand and the second nucleic acid strand anneal to each other through Watson/Crick base pairing, they are initially separate molecules before they are annealed to form a HEON. The concept of using heteroduplex oligonucleotide complexes, wherein one of the strands is bound to tocopherol or cholesterol has been described for exon skipping effects and downregulation of transcripts expression using siRNAs and gapmers (e.g., see Aoki et al. 2012. PNAS 109:13763-13768; Asada et al. 2021. Nucleic Acids Res 49:4864-4876; Asami et al. 2016. Drug Discov Ther 10(5):256-262; Asami et al. 2021. Mol Ther 29(2):838-847; Hara et al. 2018. Sci Rep 8(1):4323; Kuwahara et al. 2018. Sci Rep 8(1):4377; Nagata et al. 2021. Nat Biotechnol 39(12):1529-1536; Nishina et al. 2015. Nat Commun 6:7969; Ohyagi et al. 2021. Nat Commun 12:7344; Suzuki et al. 2021. Sci Rep 11:14237; Yoshioka et al. 2019. Nucleic Acids Res 47 (14): 7321-7332; EP2791335B1; EP2961841B1; EP3004347B1; EP3010514B1; EP35176190A1; EP3521430A1; EP3766972A1; EP3769769A1; EP3770257A1; EP3928799A1; EP3954395A1). However, the use of heteroduplex oligonucleotide complexes has not been described for RNA editing or specific deamination purposes of a single nucleotide (such as adenosine) in a target RNA molecule. RNA editing requires specific chemical modifications on the first and second nucleic acid strand. Specifics of such chemical modifications are disclosed herein.

The invention relates to a heteroduplex RNA editing oligonucleotide complex (HEON) for use in the deamination of a target adenosine present in a target RNA molecule in a cell, wherein the HEON comprises a first nucleic acid strand that is annealed to a (partly or fully) complementary second nucleic acid strand, wherein the first nucleic acid strand is complementary to a stretch of (preferably at least 8) nucleotides in the target RNA molecule that includes the target adenosine, wherein the first nucleic acid strand when it is hybridized to the target RNA molecule can recruit an enzyme with deaminase activity present in the cell allowing for the deamination of the target adenosine, wherein the nucleotide in the first nucleic acid strand that is directly opposite the target adenosine is the orphan nucleotide, wherein the orphan nucleotide does not carry a 2′-OMe substitution, and wherein the first and/or second nucleic acid strand is bound to a hydrophobic moiety. In one embodiment, the hydrophobic moiety is a lipid, a hydrophobic vitamin, or a steroid. In one embodiment, the hydrophobic vitamin is vitamin E or analog thereof. In one embodiment, the steroid is cholesterol or analog thereof. In a preferred embodiment, the invention relates to a HEON wherein the hydrophobic moiety is bound to the 5′ terminus, to the 3′ terminus, and/or to an internal position of the first nucleic acid strand and/or of the second nucleic acid strand. Preferably, it is bound to the 5′ terminus of the second nucleic acid strand. In another embodiment, the hydrophobic moiety is bound to the 3′ terminus of the second nucleic acid strand. In one embodiment, the hydrophobic moiety is bound via a cleavable linker. In one embodiment, the invention relates to a HEON according to the invention, wherein the HEON is further bound to a ligand that enables cell-specific targeting. A preferred ligand is a N-acetylgalactosamine (GalNAc) moiety or cluster that allows more efficient entry into liver cells (such as hepatocytes) through the interaction with asialoglycoprotein receptors. The skilled person is aware of a wide variety of GalNAc (cluster) varieties that are available and that may suitably be used for delivering HEONs according to the present invention to liver cells, for the treatment or amelioration of a (genetic) liver disease. Other ligands that may be used in the HEONs of the present invention to target the asialoglycoprotein receptors on liver cells are arabinogalactan, pullalan, lactobionic acid, and derivatives thereof (Warrier D U et al. Int J Biol Macromol. 2022. 207:683-699). For other cells and for other purposes other ligands may be applied and be bound to the HEONs of the present invention.

In another embodiment, the invention relates to a HEON according to the invention, wherein the first nucleic acid strand and/or the second nucleic acid strand comprises one or more nucleosides that comprise a chemical modification that is a mono- or a di-substitution at the 2′, 3′ and/or 5′ position of the ribose sugar, selected from the group consisting of: —OH; —H; —F; substituted or unsubstituted, linear or branched lower (C₁-C₁₀) alkyl, alkenyl, alkynyl, alkaryl, allyl, or aralkyl, that may be interrupted by one or more heteroatoms; —O—, S—, or N-alkyl; —O—, S—, or N-alkenyl; —O—, S—, or N-alkynyl; —O—, S—, or N-allyl; —O-alkyl-O-alkyl; -methoxy; -aminopropoxy; -methoxyethoxy; -dimethylamino oxyethoxy; and -dimethylaminoethoxyethoxy. In a preferred embodiment the orphan nucleotide in the first nucleic acid strand, when it does not have any other chemical modifications to the ribose, base, or linkage, does not comprise a 2′-OMe substitution. The 2′-OMe modification at the orphan nucleotide, when the first nucleic acid strand is in a natural configuration, generally prevents efficient RNA editing of the target adenosine. In an embodiment, the orphan nucleotide in the first nucleic acid strand has the structure of formula:

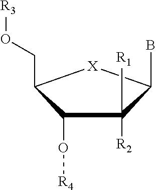

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-2(1H)-pyridone; R₁ and R₂ are both selected, independently, from H, OH, F or CH₃; R₃ is the part of the first nucleic acid strand that is 5′ of the orphan nucleotide, consisting of 7 to 30 nucleotides; and R₄ is the part of the first nucleic acid strand that is 3′ of the orphan nucleotide, consisting of 4 to 25 nucleotides. Isouracil is a uracil analog as disclosed in WO2021/071858.

In one embodiment, the first nucleic acid strand and/or the second nucleic acid strand comprises at least one phosphorothioate (PS), phosphonoacetate, methylphosphonate (MP), phosphoryl guanidine, or PNdmi internucleotide linkage. In one preferred embodiment, the first nucleic acid strand comprises a single MP linkage at linkage position-1. In another preferred embodiment, the first nucleic acid strand comprises a PNdmi linkage at the most terminal linkage at the 5′ and/or 3′ terminus. In another preferred embodiment, the first nucleic acid strand and/or the second nucleic acid strand comprises PS linkages linking the most terminal four nucleosides at the 5′ and/or 3′ end.

The HEON according to the invention comprises a first nucleic acid strand that, after entry into the cell, attaches to the target RNA molecule and forms a double-stranded nucleic acid complex with the endogenous target (pre-) mRNA and thereby can attract (recruit) an enzyme with deaminase activity. Preferably, the enzyme with deaminase activity is endogenously present in the cell in which the first nucleic acid strand performs its action, and preferably the enzyme is an endogenous ADAR enzyme, preferably a human ADAR enzyme, and even more preferably human ADAR1 or ADAR2.

The invention also relates to a HEON according to the invention, wherein the first nucleic acid strand is longer or shorter than the second nucleic acid strand. In one embodiment, the overlap between the first and second nucleic acid strands is fully complementary.

The invention also relates to a pharmaceutical composition comprising a HEON according to the invention, further comprising a pharmaceutically acceptable carrier and/or solvent. The skilled person is aware what suitable pharmaceutically acceptable carrier and/or solvents are applicable for a particular use of nucleic acid molecules and double stranded complexes such as those disclosed herein.

The invention also relates to a HEON according to the invention, or a pharmaceutical composition according to the invention, for use in the treatment or prevention of a disorder selected from the group consisting of: Hurler Syndrome, alpha-1-antitrypsin (A1AT) deficiency, (familial) hypercholesterolemia, Parkinson's disease, Rett syndrome, Stargardt Disease, Citrullinemia Type 1, autosomal recessive non-syndromic hearing loss, X-linked retinoschisis, argininosuccinate lyase deficiency, Duchenne/Becker muscular dystrophy, Non-Alcoholic Steatohepatitis (NASH), Myotonic dystrophy type I, Myotonic dystrophy type II, Huntington's disease, Usher syndrome (such as Usher syndrome type I, II, and III), Charcot-Marie-Tooth disease, Cystic fibrosis, Alzheimer's disease, albinism, Amyotrophic lateral sclerosis, Asthma, β-thalassemia, Epileptic Encephalopathy, CADASIL syndrome, Chronic Obstructive Pulmonary Disease (COPD), Distal Spinal Muscular Atrophy (DSMA), Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase deficiency, Haemophilia, Hereditary Hemochromatosis, Hereditary Cancer predisposing Syndrome, Hunter Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anaemia, Spinal Muscular Atrophy, Tay-Sachs Disease, X-linked immunodeficiency, Sturge-Weber Syndrome, and cancer, such as breast and lung cancer. Even though many disorders may be caused by a specific G to A mutation, which may result in a premature termination codon, and which may be reversed by using a HEON according to the invention, many disorders may also be the result of aberrant splicing events, loss-of-function or gain-of-function mutations, aberrant phosphorylation events, internal enzymatic cleavage events, etc, and may be treatable using a HEON according to the present invention.

In one embodiment, the invention relates to a method for the deamination of at least one target adenosine in a target RNA in a cell, the method comprising the steps of: (i) providing the cell with a HEON as defined herein; (ii) allowing annealing of the first nucleic acid strand to the target RNA in the cell to form a double stranded nucleic acid molecule; (iii) allowing an ADAR enzyme to complex with the double stranded nucleic acid molecule between the guide oligonucleotide and the target RNA and to deaminate the target adenosine in the target RNA to an inosine; and (iv) optionally identifying the presence of the deaminated nucleotide in the target RNA. In one embodiment, step (iv) comprises: a) sequencing a region of the target RNA, wherein the region comprises the position of the target adenosine; b) assessing the presence of a functional, elongated, full length and/or wild type protein when the target adenosine is in a stop codon; c) assessing, when the target RNA is pre-mRNA, whether splicing of the pre-mRNA was altered by the deamination; or d) using a functional read-out, wherein the target RNA after the deamination encodes a functional, full length, elongated and/or wild type protein.

### First and Second Nucleic Acid Strands

The first nucleic acid strand is a single-stranded oligonucleotide strand that comprises a base sequence capable of hybridizing to all or part of the transcription product of a target gene and produces an RNA editing effect on the target transcription product. The first nucleic acid strand is sometimes and herein often also referred to as the ‘RNA editing oligonucleotide’, ‘EON’, or ‘guide oligonucleotide’ that causes the specific deamination of a target adenosine present in the target RNA by recruiting an enzyme with deaminase activity (preferably an ADAR enzyme). This recruitment preferably takes place in a cell of interest and the enzyme is preferably naturally (endogenously) present in that cell. Promiscuous editing is generally prevented by chemical modification of a large part of the first nucleic acid strand, and the orphan nucleotide (the nucleotide directly opposite the target adenosine in the target RNA) and its surrounding nucleotides generally require certain very specific modifications to allow the sole deamination of the target adenosine only. The modifications of the orphan nucleotide in the nucleic acid complexes of the present invention allow the deamination of the target adenosine.

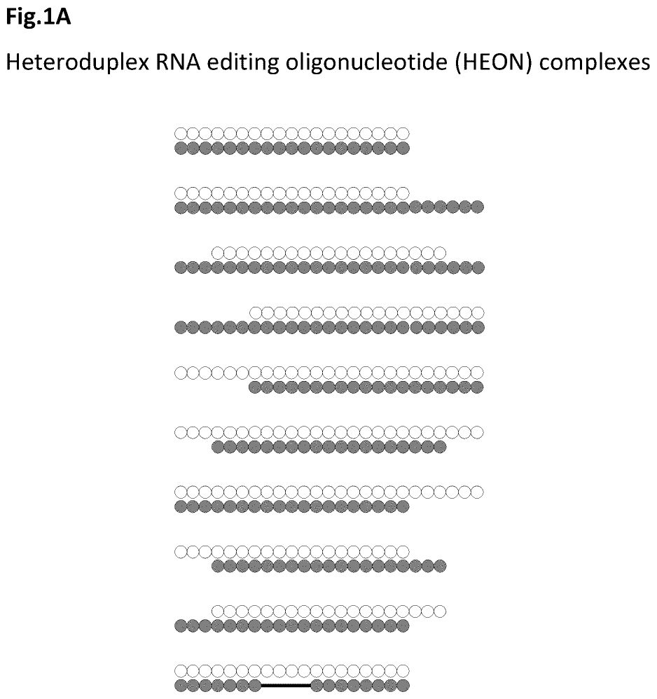

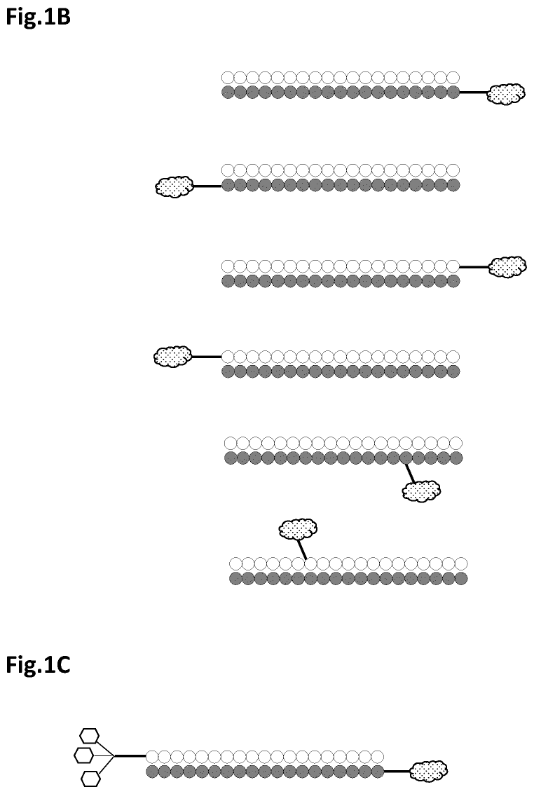

The second nucleic acid strand is a single-stranded oligonucleotide strand that comprises a base sequence that is fully or partially complementary to the first nucleic acid strand. FIG. 1 shows several (non-limiting) examples of HEONs according to the present invention. The (possible) difference in length between the first nucleic acid strand and the second nucleic acid strand is shown and the skilled person will appreciate that a variety of lengths of each of the strands is possible, resulting in differences in length of the overhang and/or overlap between first and second nucleic acid strand.

### Conjugates

The first, the second or both nucleic acid strands are preferably bound to one or more similar or dissimilar ligands such as (fragments or clusters or clusters-of-fragments of) peptides, proteins, carbohydrates, antibodies, aptamers, vitamins, natural small molecules, synthetic small molecules, drugs, hydrophobic moieties, or combinations of one or more thereof (e.g., glycolipids) at one or more of terminal or internal positions. Hydrophobic moieties may be selected from polymers, steroids, lipids, terpenes, organic molecules, vitamins, small molecule drugs, such as C₁₀ and longer optionally substituted or heteroatom-interrupted alkyl chains including lauryl, myristyl, stearyl, linolyl, oleyl, arachidyl, behenyl, lignoceryl, cerotyl, myristoleyl, palmitoleyl, sapienyl, elaidyl, vaccenyl, linolenyl, linoelaidyl, linoleyl, arachidonyl, eicosapentaenyl, erucyl, docosahexaenyl, cervoniyl, palmityl, saturated fatty acyl, unsaturated fatty acyl, ω-3 fatty acyl, ω-6 fatty acyl, ω-7 fatty acyl, ω-9 fatty acyl, multi-unsaturated fatty acyl, eicosadienyl, docosadienyl, pinolenyl, eleostearyl, mead acyl, dihomo-gamma-linolenyl, eicosatrienyl, stearidonyl, eicosatetraenyl, ozubondo acyl, sardine acyl, cervonyl, nisinyl, adrenyl, bosseopentaenyl, tridecyl, pentadecyl, margaryl, nonadecyl, heneicosyl, behenyl, tricosyl, pentacosyl, carboceryl, montanyl, nonacosyl, melissyl, hentriacontyl, lacceryl, psyllyl, geddyl, ceroplastyl, di- or trilipid constructs (such as described in WO2019/232255), adamantyl, cholesterol, farnesyl, vitamin E, vitamin A, vitamin D, vitamin K, geranylgeranyl, fluorescent labels, Cy labels, fullerenes, graphenes, pyrenyl, isoprenyl triglyceride, phospholipid, sphingolipid, glycerolipid, prenyl, leukotriene; and derivatives thereof. Preferred are hydrophobic moieties, such as a lipid, steroids, and more preferably cholesterol or analog thereof, a hydrophobic vitamins or analogs thereof or an alkyl-based moiety, preferably C16 (palmityl) or C18 (stearyl) or adamantyl or analogs thereof either directly or via one or more linkers, in a linear or cluster-like fashion. Highly preferred hydrophobic moieties are cholesterol, vitamin E and C16.

### HEON Characteristics

There is no particular restriction on the length of the first nucleic acid strand and the second nucleic acid strand, which may be both at least 8, 9, 10, 11, 12, 13, 14, or 15 nucleotides in length. Further, the length of the first nucleic acid strand and the second nucleic acid strand may be 38, 37, 36, 35, 34, 33, 32, 31, 30, 29, 28, 27, 26, 25, 24, 23, 22, 21, 20, 19, 18, 17, or 16 nucleotides in length or less. The first nucleic acid strand and the second nucleic acid strand may have the same length or have different lengths. For example, the second nucleic acid strand may be 1, 2, 3, 4, or more nucleotides shorter or longer than the first nucleic acid strand. The second nucleic acid strand may be longer than the first nucleic acid strand at the 5′ and/or 3′ terminus of the second nucleic acid strand, while the second nucleic acid strand may also be shorter than the first nucleic acid strand at the 5′ and/or the 3′ terminus of the second nucleic acid strand.

It is generally preferred that the one or more ligands, preferably a hydrophobic moiety, such as cholesterol or analog thereof, palmitate or an analogue thereof, or tocopherol or analog thereof, is bound to the second nucleic acid strand, and this may be at the 5′ terminus, at the 3′ terminus or at an internal position of the second nucleic acid strand, such as the 2′-position of a nucleoside, or the 5-position of a pyrimidine nucleobase. It is also preferred that such one or more ligands may likewise also be bound to the first nucleic acid alone, or to both first and second nucleic acids, on independent terminal or internal positions.

Independent of their lengths, the first and second nucleic acid are stretch of complementary bases may be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides, optionally interrupted with 1, 2, 3 or 4 mismatches.

When a first nucleic acid strand and a second nucleic acid strand, present and annealed to each other in a heteroduplex RNA editing oligonucleotide complex, are different in nucleotide length, or when one nucleic acid strand comprises one or more nucleotides that does not have a complementary nucleotide in the other nucleic acid strand, then the complex is often referred to as a “partial heteroduplex RNA editing oligonucleotide” complex, “partial HEON”, or “pHEON”. Generally, when the terms “HEON complex”, HEON, or “heteroduplex RNA editing oligonucleotide complex” is used herein, it encompasses also partial HEONs in which the first and second nucleic acid strands have different lengths but are still able to anneal to each other or have similar lengths but in their sequences only partially overlap. Independent of the length, the second nucleic acid strand may contain an internal loop or linker. In other words, more than one second nucleic acid strand may be linked to each other through one or more stable of cleavable linkages and/or linkers. If n second nucleic acids are coupled to each other to form one second nucleic acid molecular construct, then n first nucleic acids can be annealed to such constructs. Second nucleic acids in such construct may be of similar or different composition regarding chemistry and sequence and may accommodate up to n similar or different first nucleic acids that can be annealed. In case of n>1 first nucleic acids, at least one is an editing oligonucleotide (EON); the other(s) may be selected independently from EON, splice-switching oligonucleotide, upregulating oligonucleotide, siRNA, ss-siRNA, gapmer, saRNA, ss-saRNA, decoy nucleic acid, guide RNA, editing oligonucleotide, immunostimulatory nucleic acid, with intended biological functions and/or targets different from that of the first EON. For example, three (n=3) second nucleic acids coupled in a linear fashion to each other and bound to one or more ligands could accommodate one EON, one gapmer and one splice-switching oligonucleotide; or two EONs and one siRNA guide strand; or three EONs with similar or different targets. Such technique has been described for multiple siRNAs directed against multiple targets by e.g., Alnylam (GEMINI platform).

### Chemical Modifications

The internucleoside linkages in the first nucleic acid strand and second nucleic acid strand may be naturally occurring internucleoside linkages and/or a modified internucleoside linkages. Without limitations, at least one, at least two, or at least three internucleoside linkages from a 5′ and/or 3′ end of the first nucleic acid strand and/or the second nucleic acid strand are preferably modified internucleoside linkages. A preferred modified internucleoside linkage is a phosphorothioate linkage. In one embodiment, all internucleoside linkages of the first nucleic acid strand are modified internucleoside linkages. In one embodiment, the first nucleic acid strand comprises a PNdmi linkage linking the most terminal nucleoside at the 5′ and/or 3′ end, and the one before last nucleoside at each of these ends, respectively. A PNdmi linkage as used in the HEONs of the present invention has the structure of the following formula:

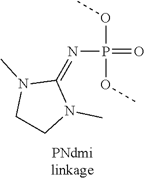

At least one (e.g., three) internucleoside linkage from the 5′ and/or 3′ end of the second nucleic acid strand may be a modified internucleoside linkage such as a phosphorothioate or phosphoramidate linkage with a high resistance to an RNase. In an embodiment, at the 5′ end and 3′ end of the second nucleic acid strand, internucleoside linkages between 2, 3, 4, 5, or 6 nucleosides from the end, to which a ligand, such as a hydrophobic moiety such as palmityl, cholesterol or tocopherol or an analog thereof is bound, may be modified internucleoside linkages such as phosphorothioate linkages.

A common limiting factor in oligonucleotide-based therapies are the oligonucleotide's ability to be taken up by the cell (when delivered per se, or ‘naked’ without applying a delivery vehicle), its biodistribution and its resistance to nuclease-mediated breakdown. The skilled person is aware, and it has been described in detail in the art, that a variety of chemical modifications can assist in overcoming such limitations. Examples of such now commonly used chemical modifications are the 2′-O-methyl(2′-OMe or 2′-O-Me), 2′-F and 2′-O-methoxyethyl(2′-MOE) modifications of the sugar and the use of phosphorothioated (PS) linkages between nucleosides. WO2020/201406 discloses the use of methylphosphonate (MP) linkage modifications at certain positions surrounding the orphan nucleotide in the first nucleic acid strand. The ribose 2′ groups in all nucleotides of the first nucleic acid strand, except for the ribose sugar moiety of the orphan nucleotide, can be independently selected from 2′-H (i.e., DNA), 2′-OH (i.e., RNA), 2′-OMe, 2′-MOE, 2′-F, or 2′-4′-linked (for instance a locked nucleic acid (LNA)), or other ribosyl 1′-substitutions, 2′ substitutions, 3′ substitutions, 4′ substitutions or 5′ substitutions. The orphan nucleotide in a first nucleic acid strand that comprises no other chemical modifications to the ribose sugar, the base, or the linkage preferably does not carry a 2′-OMe or 2′-MOE substitution but may carry a 2′-F or 2′-ara-F (FANA) substitution or may be DNA. GB 2214347.3 (unpublished) describes the modification of the 2′ position of the ribose sugar moiety of the orphan nucleotide (directly opposite the target adenosine) by a 2′,2′-disubstituted substitution such as 2′,2′-difluoro (diF), which is also applicable to the invention described here. The 2′-4′ linkage can be selected from many linkers known in the art, such as a methylene linker, amide linker, or constrained ethyl linker (cEt).

The invention relates to a composition comprising a set of two nucleic acid strands, wherein the first nucleic acid strand is sometimes referred to as the ‘antisense oligonucleotide’ and the second nucleic acid strand is sometimes referred to as the ‘sense oligonucleotide, and wherein the first and second nucleic acid strands are annealed to each other, for use in the deamination of a target nucleotide (preferably adenosine) in a target RNA, wherein the first nucleic acid strand is complementary to a stretch of nucleotides in the target RNA that includes the target adenosine, wherein the nucleotide in the first nucleic acid strand that is directly opposite the target nucleotide is the ‘orphan nucleotide’, and when the target nucleotide is an adenosine the orphan nucleotide comprises preferably a base or modified base or base analogue with a NH moiety at the position similar to the ring nitrogen (e.g., Benner's base Z). The nucleotide numbering in the first nucleic acid strand is such that the orphan nucleotide is number 0 and the nucleotide 5′ from the orphan nucleotide is number+1. Counting is further positively (+) incremented towards the 5′ end and negatively (−) incremented towards the 3′ end, wherein the first nucleotide 3′ from the orphan nucleotide is number-1. The internucleoside linkage numbering in the first nucleic acid strand is such that linkage number 0 is the linkage 5′ from the orphan nucleotide, and the linkage positions in the oligonucleotide are positively (+) incremented towards the 5′ end and negatively (−) incremented towards the 3′ end.

Preferably, the first nucleic acid strand comprises one or more (chirally pure or chirally mixed) phosphorothioate (PS) linkages. Preferably the PS linkages connect the terminal 3, 4, 5, 6, 7, or 8 nucleotides on each end of the first nucleic acid strand. Preferably, the first nucleic acid strand comprises one of more phosphoramidate (PN) linkages. Preferably, the PN linkages connect the terminal 2 nucleotides on each end of the first nucleic acid strand.

At least one (e.g., three) nucleoside from the 5′ and/or 3′ end of the second nucleic acid strand may be a modified nucleoside, such as a nucleoside comprising a 2′-F, 2′-OMe, LNA, inverted dT or 2′-MOE substitution, preferably a modification that increases resistance towards RNase breakdown.

A nucleoside in the first nucleic acid strand and the second nucleic acid strand may be a natural nucleoside (deoxyribonucleoside or ribonucleoside) or a non-natural nucleoside. It is noted that for RNA editing, in which double-stranded RNA is generally the substrate for enzymes with deamination activity (such as ADARs), ribonucleosides are considered ‘natural’, while deoxyribonucleosides may then be, for the sake of argument, considered as non-natural, or modified, simply because DNA is not present in the RNA-RNA double stranded substrate configurations. The skilled person appreciates that when the nucleotide has a natural ribose moiety, it may still be non-naturally modified in the base and/or the linkage.

The base sequence of the first nucleic acid strand herein is complementary to all or part of the base sequence of a target transcription product including the target adenosine that is to be deaminated to an inosine, and therefore can anneal (or hybridize) to the target transcription product. The complementarity of a base sequence can be determined by using a BLAST program or the like. Those skilled in the art can easily determine the conditions (temperature, salt concentration, and the like) under which two strands can be hybridized, taking into consideration the complementarity between the strands.

The first nucleic acid according to the present invention, in contrast to what has been described for gapmers and their vulnerability towards RNase breakdown and the use of such gapmers in double-stranded complexes (see for instance EP 3954395 A1), does not comprise a stretch of DNA nucleotides which would make target sequence (or the second nucleic acid strand) a target for RNase-mediated breakdown. Preferably, the first nucleic acid strand does not comprise four or more consecutive DNA nucleotides anywhere within its sequence. In an embodiment, the first nucleic acid strand is composed of as much (chemically) modified nucleotides as possible to enhance the resistance towards RNase-mediated breakdown, while at the same time being as efficient as possible in producing an RNA editing effect. This means that the orphan nucleotide and several other nucleotides within the first nucleic acid strand may be DNA, but also that there is no stretch of four or more consecutive DNA nucleotides within the first nucleic acid strand.

In one embodiment, the second nucleic acid strand may comprise at least 4, 5, 6, 7, 8, 9, 10, 11, 12, 13, 14, 15, 16, 17, 18, or 19 consecutive deoxyribonucleosides. The consecutive deoxyribonucleosides in the second nucleic acid strand of the present invention are preferably linked by naturally occurring internucleoside linkages, namely phosphodiester linkages. In one embodiment, the one, two or three nucleosides on either end of the second nucleic acid strand are non-natural nucleosides and are preferably modified by 2′-substitutions such as 2′-OMe, 2′-F, LNA or 2′-MOE substitutions. The modified nucleobase may also be a 5-methyl cytosine, or a 5-methyl uridine. In one embodiment, the two, three, or four nucleosides on either end of the second nucleic acid strand are linked by a non-natural internucleoside linkage such as a phosphorothioate linkage, and the remainder of the nucleosides within the second nucleic acid strand are preferably deoxyribonucleosides and the remainder of the linkages within the second nucleic acid strand are preferably phosphodiester linkages.

The first nucleic acid strand according to the present invention is not a gapmer. A gapmer is in principle a single-stranded nucleic acid consisting of a central region (DNA gap region with at least four consecutive deoxyribonucleotides) and wing regions positioned directly at the 5′ end (5′ wing region) and the 3′ end (3′ wing region) thereof. In contrast, the first nucleic acid strand according to the invention may be any oligonucleotide that produces an RNA editing effect in which a target adenosine in a target RNA molecule is deaminated to an inosine, and accordingly is resistant to RNase-mediated breakdown as much as possible to yield this effect. Any RNA editing producing oligonucleotide, or EON, described in the art may serve as the first nucleic acid according to the present invention in which it is annealed to a second nucleic acid strand that is bound to a ligand, such as a hydrophobic moiety such as palmityl or an analog thereof, cholesterol or analog thereof, or tocopherol or analog thereof, to ultimately form a (partial) heteroduplex RNA editing oligonucleotide (HEON) complex. This means that EONs that have already been described in the art or that can be deduced or derived from EONs described in the art, may be in a HEON complex according to the present invention.

Preferably, the second nucleic acid strand of the HEONs of the present invention is bound to a hydrophobic moiety as disclosed herein, such as palmityl or an analog thereof, cholesterol or analog thereof, or tocopherol or analog thereof. In case the second nucleic acid strand is bound to one hydrophobic moiety, it is preferably bound to its 5′ terminus. In case a hydrophobic moiety is bound to the 5′ terminus as well as to the 3′ terminus, such hydrophobic moieties may the same or different. The hydrophobic moiety bound to the second nucleic acid strand may be bound directly, or indirectly mediated by another substance. When the hydrophobic moiety is bound directly, it is sufficient if the moiety is bound to the second nucleic acid strand via a covalent bond, an ionic bond, a hydrogen bond, or the like. When the hydrophobic moiety is bound indirectly to the second nucleic acid strand, it may be bound via a linking group (a linker). The linker may be a cleavable or an uncleavable linker. A cleavable linker refers to a linker that can be cleaved under physiological conditions, for example, in a cell or an animal body (e.g., a human body). A cleavable linker is selectively cleaved by an endogenous enzyme such as a nuclease, or by physiological circumstances specific to parts of the body or cell, such as pH or reducing environment (such as glutathione concentrations). Examples of a cleavable linker comprise, but is not limited to, an amide, an ester, one or both esters of a phosphodiester, a phosphoester, a carbamate, and a disulfide bond, as well as a natural DNA linker. Cleavable linkers also include self-immolative linkers. An uncleavable linker refers to a linker that is not cleaved under physiological conditions, or very slowly compared to a cleavable linker, for example, in a phosphorothioate linkage, modified or unmodified deoxyribonucleosides linked by a phosphorothioate linkage, a spacer connected through a phosphorothioate bond and a linker consisting of modified or unmodified ribonucleosides. There is no particular restriction on the chain length, when a linker is a nucleic acid such as DNA, or an oligonucleotide. However, it may be usually from 2 to 20 bases in length, from 3 to 10 bases in length, or from 4 to 6 bases in length. There is no particular restriction on the length or composition of a spacer that is connects the ligand and the oligonucleotide, and may include for example ethylene glycol, TEG, HEG, alkyl chains, propyl, 6-aminohexyl, dodecyl.

The invention also relates to a pharmaceutical composition comprising the HEON according to the invention, and further comprising a pharmaceutically acceptable carrier and/or other additive and may be dissolved in a pharmaceutically acceptable organic solvent, or the like. Dosage forms in which the HEON or the pharmaceutical composition are administered may depend on the disorder to be treated and the tissue that needs to be targeted and can be selected according to common procedures in the art. The pharmaceutical compositions may be administered by a single-dose administration or by multiple dose administration. It may be administered daily or at appropriate time intervals, which may be determined using common general knowledge in the field and may be adjusted based on the disorder and the efficacy of the active ingredient.

Preferably, the first nucleic acid strand comprises at least one nucleotide with a sugar moiety that comprises a 2′-OMe modification. Preferably, the first nucleic acid strand comprises at least one nucleotide with a sugar moiety that comprises a 2′-MOE modification. Preferably, the first nucleic acid strand comprises at least one nucleotide with a sugar moiety that comprises a 2′-F modification. In one embodiment, the orphan nucleotide carries a 2′-H in the sugar moiety and is therefore referred to as a DNA nucleotide. In one embodiment, the orphan nucleotide carries a 2′-F in the sugar moiety. In one embodiment, the orphan nucleotide carries a 2′-2′-difuoro (diF) substitution in the sugar moiety. In one embodiment, the orphan nucleotide carries a 2′-F and a 2′-C-methyl in the sugar moiety. In one embodiment, the orphan nucleotide comprises a 2′-F in the arabinose configuration (FANA) in the sugar moiety. In one embodiment, the first nucleic acid strand is an antisense oligonucleotide that can form a double stranded nucleic acid complex with a target RNA molecule, wherein the double stranded nucleic acid complex is capable of recruiting an adenosine deaminating enzyme for deamination of a target adenosine in the target RNA molecule, wherein the nucleotide in the first nucleic acid strand that is opposite the target adenosine is the orphan nucleotide, and wherein the orphan nucleotide has the following structure:

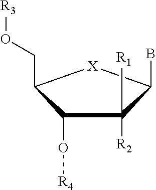

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-2(1H)-pyridone; R₁ and R₂ are both selected, independently, from H, OH, F or CH₃; R₃ is the part of the first nucleic acid strand that is 5′ of the orphan nucleotide, consisting of 7 to 30 nucleotides; and R₄ is the part of the first nucleic acid strand that is 3′ of the orphan nucleotide, consisting of 4 to 25 nucleotides. The nucleotide 3′ and/or 5′ from the orphan nucleotide may be DNA, more preferably the nucleotide at the 3′ (position-1).

In one embodiment, the first nucleic acid strand comprises at least one methylphosphonate (MP) internucleoside linkage according to the following structure:

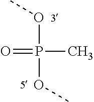

A preferred position for an MP linkage in a first nucleic acid strand present in a HEON according to the invention is linkage position-1, thereby connecting the nucleoside at position-1 with the nucleoside at position-2, although other positions for MP linkages are not explicitly excluded.

Preferably, the first nucleic acid strand comprises at least one nucleotide with a sugar moiety that comprises a 2′-fluoro (2′-F) modification. A preferred position for the nucleotide that carries a 2′-F modification is position-3 in the first nucleic acid strand, which may be present together with an identical 2′ modification in the orphan nucleotide as discussed above.

In an embodiment, the first nucleic acid strand comprises at least one phosphonoacetate or phosphonoacetamide internucleoside linkage.

In an embodiment, the first nucleic acid strand comprises at least one nucleotide comprising a locked nucleic acid (LNA) ribose modification, or an unlocked nucleic acid (UNA) ribose modification. In an embodiment, the first nucleic acid strand comprises at least one nucleotide comprising a threose nucleic acid (TNA) ribose modification.

The skilled person knows that an oligonucleotide, such as a first and second nucleic acid strand as outlined herein, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide.

A modification in the pentose sugar is therefore often referred to as a “scaffold modification”. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the nucleic acid strands present in the HEON of the present invention are disclosed in WO2020/154342, WO2020/154343, and WO2020/154344.

In one embodiment, the first and/or second nucleic acid strand in the HEON of the present invention may comprise one or more nucleotides carrying a 2′-MOE ribose modification. Also, in one embodiment, the first and/or second nucleic acid strand in the HEON comprises one or more nucleotides not carrying a 2′-MOE ribose modification, and wherein the 2′-MOE ribose modifications are at positions that do not prevent the enzyme with adenosine deaminase activity from deaminating the target adenosine. In another embodiment, the first and/or second nucleic acid strand in the HEON comprises 2′-O-Me ribose modifications at the positions that do not comprise a 2′-MOE ribose modification, and/or wherein the oligonucleotide comprises deoxynucleotides at positions that do not comprise a 2′-MOE ribose modification. In one embodiment the first and/or second nucleic acid strand in the HEON comprises one or more nucleotides comprising a 2′ position comprising a 2′-MOE, 2′-O-Me, 2′-OH, 2′-deoxy, TNA, 2′-fluoro (2′-F), 2′,2′-difluoro (diF) modification, 2′-fluoro-2′-C-methyl modification, or a 2′-4′-linkage (i.e., a bridged nucleic acid such as a locked nucleic acid (LNA or examples mentioned in e.g. WO2018/007475)). In another embodiment, other nucleic acid monomer that are applied are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications are known in the art. Further examples are disclosed in further detail in WO2016/097212, WO2017/220751, WO2018/041973, WO2018/134301, WO2019/219581, WO2019/158475, and WO2022/099159 for instance. In all cases, the modifications should be compatible with editing such that the first nucleic acid strand fulfils its role as an editing oligonucleotide. Where a monomer comprises an unlocked nucleic acid (UNA) ribose modification, that monomer can have a 2′ position comprising the same modifications discussed above, such as a 2′-MOE, a 2′-O-Me, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as a locked nucleic acid (LNA)).

A base, sometimes called a nucleobase, is generally adenine, cytosine, guanine, thymine or uracil, or a derivative thereof. A base, sometimes called a nucleobase, is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen. The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy) ribosyl sugar.

The nucleobases in a first and/or second nucleic acid strand in a HEON of the present invention can be adenine, cytosine, guanine, thymine, or uracil or any other moiety able to interact with another nucleobase through H-bonds, polarized bonds (such as CF) or aromatic electronic interactions. The nucleobases at any position in the nucleic acid strand can be a modified form of adenine, cytosine, guanine, or uracil, such as hypoxanthine (the nucleobase in inosine), pseudouracil, pseudocytosine, isouracil, N3-glycosylated uracil, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose).

In an embodiment, the nucleotide analog is an analog of a nucleic acid nucleotide. In an embodiment, the nucleotide analog is an analog of adenosine, guanosine, cytidine, thymidine, uridine, deoxyadenosine, deoxyguanosine, deoxycytidine, deoxythymidine or deoxyuridine. In an embodiment, the nucleotide analog is not guanosine or deoxyguanosine. In an embodiment, the nucleotide analog is not a nucleic acid nucleotide. In an embodiment, the nucleotide analog is not adenosine, guanosine, cytidine, thymidine, uridine, deoxyadenosine, deoxyguanosine, deoxycytidine, deoxythymidine or deoxyuridine.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate (PS), such a moiety is still referred to as the backbone linkage of the monomer. This is referred to as a “backbone linkage modification”. In general terms, the backbone of an oligonucleotide comprises alternating scaffolds and backbone linkages.

First and/or second strand nucleic acids according to the invention can comprise linkage modifications. A linkage modification can be, but not limited to, a modified version of the phosphodiester present in RNA, such as phosphorothioate (PS), chirally pure phosphorothioate, (R)-phosphorothioate, (S)-phosphorothioate, methyl phosphonate (MP), chirally pure methyl phosphonate, (R)-methyl phosphonate, (S)-methyl phosphonate, phosphoryl guanidine (such as PNdmi), chirally pure phosphoryl guanidine, (R)-phosphoryl guanidine, (S)-phosphoryl guanidine, phosphorodithioate (PS2), phosphonacetate (PACE), phosphonoacetamide (PACA), thiophosphonoacetate, thiophosphonoacetamide, methyl phosphorohioate, methyl thiophosphonate, phosphorothioate prodrug, alkylated phosphorothioate, H-phosphonate, ethyl phosphate, ethyl phosphorothioate, boranophosphate, boranophosphorothioate, methyl boranophosphate, methyl boranophosphorothioate, methyl boranophosphonate, methyl boranophosphothioate, phosphate, phosphotriester, aminoalkylphosphotriester, and their derivatives. Another modification includes phosphoramidite, phosphoramidate, N3′→P5′ phosphoramidate, phosphorodiamidate, phosphorothiodiamidate, sulfamate, diethylenesulfoxide, amide, sulfonate, siloxane, sulfide, sulfone, formacetyl, alkenyl, methylenehydrazino, sulfonamide, triazole, oxalyl, carbamate, methyleneimino (MMI), and thioacetamide nucleic acid (TANA); and their derivatives. Various salts, mixed salts and free acid forms are also included, as well as 3′→3′ and 2′→5′ linkages.

Again, in all cases, the modifications should be compatible with editing such that the first nucleic acid strand fulfils its role as an EON that can, when attached to its target sequence recruit an adenosine deaminase enzyme. In all aspects of the invention, the enzyme with adenosine deaminase activity is preferably ADAR1, ADAR2, or ADAT. In a highly preferred embodiment, the AON is an RNA editing oligonucleotide that targets a pre-mRNA or an mRNA, wherein the target nucleotide is an adenosine in the target RNA, wherein the adenosine is deaminated to an inosine, which is being read as a guanosine by the translation machinery. The invention also relates to a pharmaceutical composition comprising the HEON as characterized herein, and a pharmaceutically acceptable carrier.

Other chemical modifications of the HEON according to the invention include the substitution of one or more than one of any of the hydrogen atoms with deuterium or tritium, examples of which can be found in e.g., WO2014/022566 or WO2015/011694.

The invention relates to a HEON according to the invention, or a pharmaceutical composition comprising a HEON according to the invention, for use in the treatment or prevention of a genetic disorder, preferably selected from the group consisting of: Hurler Syndrome, alpha-1-antitrypsin (A1AT) deficiency, (familial) hypercholesterolemia, Parkinson's disease, Rett syndrome, Stargardt Disease, Citrullinemia Type 1, autosomal recessive non-syndromic hearing loss, X-linked retinoschisis, argininosuccinate lyase deficiency, Duchenne/Becker muscular dystrophy, Non-Alcoholic Steatohepatitis (NASH), Myotonic dystrophy type I, Myotonic dystrophy type II, Huntington's disease, Usher syndrome (such as Usher syndrome type I, II, and III), Charcot-Marie-Tooth disease, Cystic fibrosis, Alzheimer's disease, albinism, Amyotrophic lateral sclerosis, Asthma, β-thalassemia, Epileptic Encephalopathy, CADASIL syndrome, Chronic Obstructive Pulmonary Disease (COPD), Distal Spinal Muscular Atrophy (DSMA), Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase deficiency, Haemophilia, Hereditary Hemochromatosis, Hereditary Cancer predisposing Syndrome, Hunter Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anaemia, Spinal Muscular Atrophy, Tay-Sachs Disease, X-linked immunodeficiency, Sturge-Weber Syndrome, and cancer, such as breast and lung cancer.

In one embodiment, the invention relates to a HEON comprising a first nucleic acid strand and a second nucleic acid strand as outlined above, wherein the first nucleic acid strand is capable of forming a double stranded nucleic acid complex with a target RNA molecule, for use in the treatment of a genetic disorder, wherein the then-formed double stranded nucleic acid complex can recruit an adenosine deaminating enzyme for deamination of a target adenosine in the target RNA molecule.

In one embodiment, the invention relates to an HEON comprising a first nucleic acid strand and a second nucleic acid strand as outlined above, wherein the first nucleic acid strand is capable of forming a double stranded nucleic acid complex with a target RNA molecule, for use in the treatment of a disease, disorder or condition, wherein the then-formed double stranded nucleic acid complex is capable of recruiting an adenosine deaminating enzyme for deamination of a target adenosine in the target RNA molecule, and wherein the disorder is not caused by a mutation, but wherein the use is for a gain-of-function or loss-of-function purpose to alleviate, treat, prevent, or ameliorate a disease, disorder or condition. In another embodiment, the invention relates to a method of treating a subject, preferably a human subject in need thereof, wherein the subject suffers from a genetic disorder caused by a mutation resulting in a premature termination codon.

### Definitions

The term ‘nucleoside’ refers to the nucleobase linked to the (deoxy) ribosyl sugar, without phosphate groups. A ‘nucleotide’ is composed of a nucleoside and one or more phosphate groups. The term ‘nucleotide’ thus refers to the respective nucleobase-(deoxy) ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a threose nucleic acid (TNA), a nucleotide including a linker comprising a phosphodiester, phosphonoacetate, phosphotriester, PS, phosphoro(di)thioate, MP, methyl thiophosphonate, phosphoramidate linkages, and the like. Sometimes the terms adenosine and adenine, guanosine and guanine, cytidine and cytosine, uracil and uridine, thymine and thymidine/uridine, inosine and hypoxanthine, are used interchangeably to refer to the corresponding nucleobase on the one hand, and the nucleoside or nucleotide on the other. Thymine (T) is also known as 5-methyluracil (m⁵U) and is an uracil (U) derivative; thymine, 5-methyluracil and uracil can be interchanged throughout the document text. Likewise, thymidine is also known as 5-methyluridine and is a uridine derivative; thymidine, 5-methyluridine and uridine can be interchanged throughout the document text.

Sometimes the terms nucleobase, nucleoside and nucleotide are used interchangeably, unless the context clearly requires differently, for instance when a nucleoside is linked to a neighbouring nucleoside and the linkage between these nucleosides is modified. As stated above, a nucleotide is a nucleoside plus one or more phosphate groups. The terms ‘ribonucleoside’ and ‘deoxyribonucleoside’, or ‘ribose’ and ‘deoxyribose’ are as used in the art. Whenever reference is made to a first or second nucleic acid strand, an oligonucleotide, oligo, ON, ASO, oligonucleotide composition, antisense oligonucleotide, AON, (RNA) editing oligonucleotide, EON, and RNA (antisense) oligonucleotide both oligoribonucleotides and deoxyoligoribonucleotides are meant unless the context dictates otherwise. Potentially the oligonucleotide of the first nucleic acid strand and/or the second nucleic acid strand may completely lack RNA or DNA nucleotides (as they appear in nature) and may consist completely of modified nucleotides. Whenever reference is made to an ‘oligoribonucleotide’ it may comprise the bases A, G, C, U or I. Whenever reference is made to a ‘deoxyoligoribonucleotide’ it may comprise the bases A, G, C, T or I. However, a first and/or second nucleic acid strand in a HEON of the present invention may comprise a mix of ribonucleosides and deoxyribonucleosides. When a deoxyribonucleoside is used, hence without a modification at the 2′ position of the sugar, the nucleotide is often abbreviated to dA. dC, dG or T in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

Whenever reference is made to nucleotides in the oligonucleotide, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included; when reference is made to adenine, N6-methyladenine, 8-oxo-adenine, 2,6-diaminopurine and 7-methyladenine are included; when reference is made to uracil, dihydrouracil, isouracil, N3-glycosylated uracil, pseudouracil, 5-methyluracil, N1-methylpseudouracil, 4-thiouracil and 5-hydroxymethyluracil are included; when reference is made to guanine, 1-methylguanine, 7-methylguanosine, N2,N2-dimethylguanosine, N2,N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-O-methyl, are included, as well as other modifications, including 2′-4′ bridged variants. Whenever reference is made to oligonucleotides, linkages between two mononucleotides may be phosphodiester linkages as well as modifications thereof, including, phosphonoacetate, phosphodiester, phosphotriester, PS, phosphoro(di)thioate, MP, phosphoramidate linkers, phosphoryl guanidine, thiophosphoryl guanidine, sulfono phosphoramidate and the like.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., x±10%. The word ‘substantially’ does not exclude ‘completely’, e.g., a composition which is ‘substantially free from Y’ may be completely free from Y. Where relevant, the word ‘substantially’ may be omitted from the definition of the invention.

The term “complementary” as used herein refers to the fact that the first nucleic acid strand hybridizes under physiological conditions to the second nucleic acid strand or to the target RNA sequence. The term does not mean that each nucleotide in a nucleic acid strand has a perfect pairing with its opposite nucleotide in the opposite sequence. In other words, while a first nucleic acid strand may be complementary to a target sequence, there may be mismatches, wobbles and/or bulges between the first nucleic acid strand and the target sequence, while under physiological conditions that first nucleic acid strand still hybridizes to the target sequence such that the cellular RNA editing enzymes can edit the target adenosine. The term “substantially complementary” therefore also means that despite the presence of the mismatches, wobbles, and/or bulges, the first nucleic acid strand has enough matching nucleotides between the first nucleic acid strand and target sequence that under physiological conditions the first nucleic acid strand hybridizes to the target RNA. As shown herein, a first nucleic acid strand may be complementary, but may also comprise one or more mismatches, wobbles and/or bulges with the target sequence, if under physiological conditions the first nucleic acid strand is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity. The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C—C, U—U pairs. In some embodiments first nucleic acid strands of the present invention comprise fewer than four mismatches with the target sequence, for example 0, 1 or 2 mismatches. Wobble base pairs are G-U, I—U, I-A, and I-C base pairs. Although a G: G pairing would be considered a mismatch, that does not necessarily mean that the interaction is unstable, which means that the term ‘mismatch’ may be somewhat outdated based on the current invention where a Hoogsteen base-pairing may be seen as a mismatch based on the origin of the nucleotide but still be relatively stable. An isolated G: G pairing in duplex RNA can for instance be quite stable, but still be defined as a mismatch. In a preferred embodiment, the first nucleic acid strand has no mismatches with the second nucleic acid strand, while it may have mismatches with the target RNA sequence that comprises the target adenosine.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity, as discussed herein. The exact mutation does not have to be the target for the RNA editing; it may be that a neighbouring or nearby adenosine in the (splice) mutation is the target nucleotide, conversion of which to I fixes the splice mutation back to a normal state. The skilled person is aware of methods to determine whether normal splicing is restored, after RNA editing of the adenosine within the splice mutation site or area.

A first nucleic acid strand in a HEON according to the present invention may be chemically modified almost in its entirety, for example by providing nucleotides with a 2′-O-Me modification, 2′-F, and/or with a 2′-MOE sugar moiety. However, the orphan nucleotide in a first nucleic acid strand is preferably a cytidine or a cytidine analog and/or in one embodiment comprises a diF modification at the 2′ position of the sugar, and in yet a further embodiment, at least one and in another embodiment both the two neighbouring nucleotides flanking the orphan nucleotide do not comprise a 2′-O-Me modification. Complete modification wherein all nucleotides of the first nucleic acid strand hold a 2′-O-Me modification, with natural bases, results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-O-Me group (at least when there are no other chemical substitutions or modifications within the nucleotide), or by providing a guanine or adenine as opposing base, as these two nucleobases are also able to reduce editing of the opposing adenosine. Various chemistries and modification are known in the field of oligonucleotides that can be readily used in accordance with the invention. The regular internucleosidic linkages between the nucleotides may be altered by mono- or di-thioation of the phosphodiester bonds to yield phosphorothioate esters or phosphorodithioate esters, respectively. Other modifications of the internucleosidic linkages are possible, including amidation and peptide linkers. In an embodiment, the first and/or second nucleic acid strand in a HEON of the present invention comprise 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate adenosines in a non-discriminative manner, reacting with any adenosine it encounters. The specificity of hADAR1 and 2 can be increased by introducing chemical modifications and/or ensuring several mismatches in the dsRNA, which presumably help to position the dsRNA binding domains in a way that has not been clearly defined yet. Additionally, the deamination reaction itself can be enhanced by providing an oligonucleotide that comprises a mismatch opposite the adenosine to be edited. Following the instructions in the present application, those of skill in the art will be capable of designing the complementary portion of the oligonucleotide according to their needs.

The RNA editing protein present in the cell that is of most interest to be used with HEONs of the present invention is human ADAR2. It will be understood by a person having ordinary skill in the art that the extent to which the editing entities inside the cell are redirected to other target sites may be regulated by varying the affinity of the first nucleic acid strand for the recognition domain of the editing molecule. The exact modification may be determined through some trial and error and/or through computational methods based on structural interactions between the first nucleic acid strand and the recognition domain of the editing molecule. In addition, or alternatively, the degree of recruiting and redirecting the editing entity resident in the cell may be regulated by the dosing and the dosing regimen of the HEON. This is something to be determined by the experimenter (in vitro) or the clinician, usually in phase I and/or II clinical trials.

The invention concerns the modification of target RNA sequences in eukaryotic, preferably metazoan, more preferably mammalian, most preferably human cells. The invention can be used with cells from any organ e.g., skin, lung, heart, kidney, liver, pancreas, gut, muscle, gland, eye, brain, blood, and the like. The invention is particularly suitable for modifying sequences in cells, tissues or organs implicated in a diseased state of a (human) subject. The cell can be located in vitro, ex vivo or in vivo. One advantage of the invention is that it can be used with cells in situ in a living organism, but it can also be used with cells in culture. In some embodiments cells are treated ex vivo and are then introduced into a living organism (e.g., re-introduced into an organism from whom they were originally derived). The invention can also be used to edit target RNA sequences in cells from a transplant or within a so-called organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant. The cell to be treated will generally have a genetic mutation. The mutation may be heterozygous or homozygous. The invention will typically be used to modify point mutations, such as N to A mutations, wherein N may be G, C, U (on the DNA level T), preferably G to A mutations, or N to C mutations, wherein N may be A, G, U (on the DNA level T), preferably U to C mutations.

Without wishing to be bound by theory, the RNA editing through hADAR2 is thought to take place on primary transcripts in the nucleus, during transcription or splicing, or in the cytoplasm, where e.g., mature mRNA, miRNA or ncRNA can be edited.

Many genetic diseases are caused by G to A mutations, and these are preferred target diseases because adenosine deamination at the mutated target adenosine will reverse the mutation to a codon giving rise to a functional, full length wild type protein, especially when it concerns PTCs.

It should be clear, that targeted editing according to the invention can be applied to any adenosine, whether it is a mutated or a wild-type nucleotide. For example, editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether to prevent, delay or treat disease or for any other purpose, in medicine or biotechnology, as diagnostic, prophylactic, therapeutic, research tool or otherwise, are encompassed by the present invention.

The amount of HEON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change. It is possible that higher doses of first nucleic acid strands could compete for binding to an ADAR within a cell, thereby depleting the amount of the entity, which is free to take part in RNA editing, but routine dosing trials will reveal any such effects for a given HEON and a given target.

One suitable trial technique involves delivering the HEON to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained, and future delivery can be performed without needing to take biopsy samples. A method of the invention can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, the change may be assessed on the level of the protein (length, glycosylation, function, or the like), or by some functional read-out, such as a (n) (inducible) current, when the protein encoded by the target RNA sequence is an ion channel, for example.

After RNA editing has occurred in a cell, the modified RNA can become diluted over time, for example due to cell division, limited half-life of the edited RNAs, etc. Thus, in practical therapeutic terms a method of the invention may involve repeated delivery of a HEON until enough target RNAs have been modified to provide a tangible benefit to the patient and/or to maintain the benefits over time.

HEONs of the invention are particularly suitable for therapeutic use, and so the invention provides a pharmaceutical composition comprising an HEON of the invention and a pharmaceutically acceptable carrier. In some embodiments of the invention the pharmaceutically acceptable carrier can simply be a saline solution. This can usefully be isotonic or hypotonic, particularly for pulmonary delivery. The invention also provides a delivery device (e.g., syringe, inhaler, nebuliser) which includes a pharmaceutical composition of the invention.

The invention also provides an HEON of the invention for use in a method for making a change in a target RNA sequence in a mammalian, preferably a human cell, as described herein. Similarly, the invention provides the use of a HEON of the invention in the manufacture of a medicament for making a change in a target RNA sequence in a mammalian, preferably a human cell, as described herein.

The invention also relates to a method for the deamination of at least one specific target adenosine present in a target RNA sequence in a cell, the method comprising the steps of: providing the cell with a HEON according to the invention; allowing uptake by the cell of the HEON; allowing annealing of the first nucleic acid strand to the target RNA molecule; allowing a mammalian ADAR enzyme comprising a natural dsRNA binding domain as found in the wild type enzyme to deaminate the target adenosine in the target RNA molecule to an inosine; and optionally identifying the presence of the inosine in the RNA sequence.

In a preferred aspect, depending on the ultimate deamination effect of A to I conversion, the identification step comprises: sequencing the target RNA; assessing the presence of a functional, elongated, full length and/or wild type protein; assessing whether splicing of the pre-mRNA was altered by the deamination; or using a functional read-out, wherein the target RNA after the deamination encodes a functional, full length, elongated and/or wild type protein. Because the deamination of the adenosine to an inosine may result in a protein that is no longer suffering from the mutated A at the target position, the identification of the deamination into inosine may also be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. A very suitable manner to identify the presence of an inosine after deamination of the target adenosine is of course RT-PCR and sequencing, using methods that are well-known to the person skilled in the art.

The HEON according to the invention is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g., through nebulization), intranasally, orally, by injection or infusion, intravenously, subcutaneously, intradermally, intracranially, intravitreally, intramuscularly, intra-tracheally, intra-peritoneally, intrarectally, intrathecally, intra-cisterna magna, parenterally, and the like. Administration may be in solid form, in the form of a powder, a pill, a gel, an eye-drop, a solution, a slow-release formulation, or in any other form compatible with pharmaceutical use in humans.

In one embodiment, a method according to the invention comprises the steps of administering to the subject a HEON or pharmaceutical composition according to the invention, allowing the formation of a double stranded nucleic acid complex of the first nucleic acid strand with its specific complementary target nucleic acid molecule in a cell in the subject; allowing the engagement of an endogenous present adenosine deaminating enzyme, such as ADAR2; and allowing the enzyme to deaminate the target adenosine in the target nucleic target molecule to an inosine, thereby alleviating, preventing or ameliorating the genetic disease. The genetic diseases that may be treated according to this method are preferably, but not limited to the genetic diseases listed herein, and any other disease in which deamination of a specific adenosine would be beneficial for a patient in need thereof.

RNA editing molecules present in the cell will usually be proteinaceous in nature, such as the ADAR enzymes found in metazoans, including mammals. Preferably, the cellular editing entity is an enzyme, more preferably an adenosine deaminase or a cytidine deaminase, still more preferably an adenosine deaminase. These are enzymes with ADAR activity. The ones of most interest are the human ADARs, hADAR1 and hADAR2, including any isoforms thereof. RNA editing enzymes known in the art, for which oligonucleotide constructs according to the invention may conveniently be designed, include the adenosine deaminases acting on RNA (ADARs), such as hADAR1 and hADAR2 in humans or human cells and cytidine deaminases. It is known that hADAR1 exists in two isoforms; a long 150 kDa interferon inducible version and a shorter, 100 kDa version, that is produced through alternative splicing from a common pre-mRNA. Consequently, the level of the 150 kDa isoform available in the cell may be influenced by interferon, particularly interferon-gamma (IFN-γ). hADAR1 is also inducible by TNF-α. This provides an opportunity to develop combination therapy, whereby IFN-γ or TNF-α and AONs according to the invention are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the first nucleic acid strand for the recognition domain of the editing molecule.

### EXAMPLES

### Example 1. In Vivo a to I Editing in mApp Target RNA in Mouse Retina Using Heteroduplex RNA Editing Oligonucleotide Complexes (HEONs)

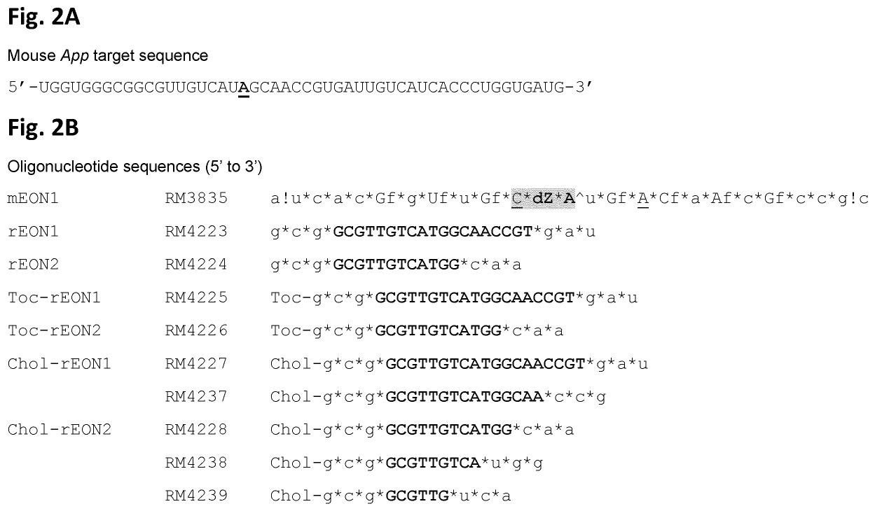

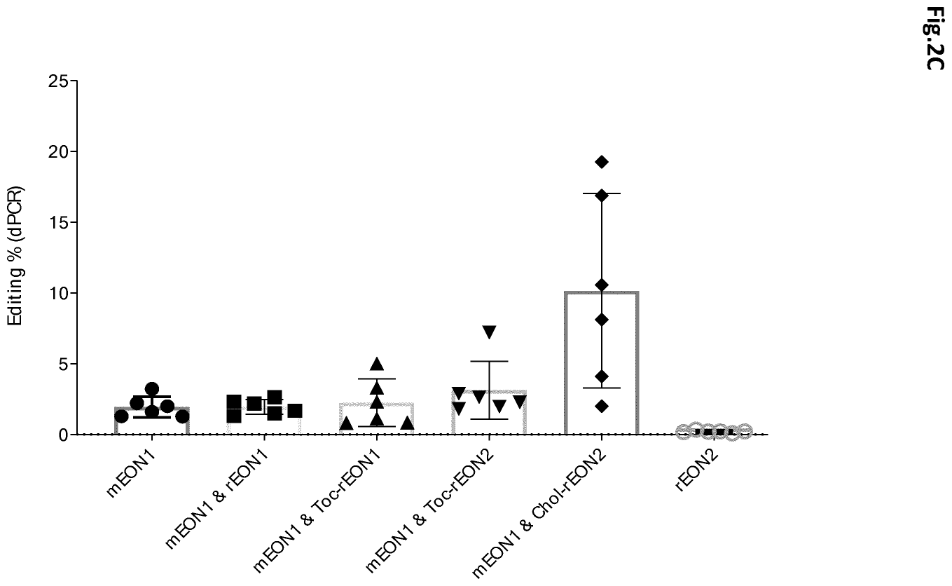

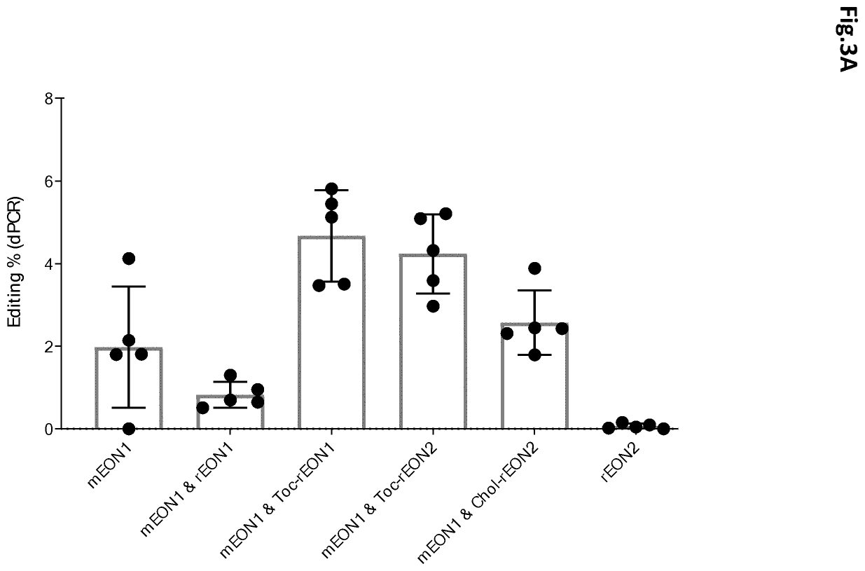

The inventors wondered whether the use of heteroduplex oligonucleotide compositions in which an RNA editing oligonucleotide (EON) acting as a first nucleic acid strand that is complementary to a target RNA molecule, would be beneficial in achieving RNA editing in an in vivo model. For this, a set of oligonucleotides were generated that would target a specific adenosine in the mouse APP transcript. FIG. 2A shows part of the sequence of the mouse App (mApp) gene product (RNA), in which the target adenosine is underlined and given in bold. FIG. 2B shows the oligonucleotide (mEON1) that targets the mApp target RNA molecule and comprises a variety of chemical modifications to enhance editing and to prevent nuclease breakdown in vivo. The specific modifications are provided in the legend to the figure. The mEON1 oligonucleotide acts as the first nucleic acid strand in HEONs according to the invention that is disclosed herein. The mEON1 first nucleic acid strand was combined with a variety of sense oligonucleotides that would then act as the second nucleic acid strand in the HEON. Two lengths were initially tested with rEON1 that is 100% complementary to mEON1 over its entire length of 25 nucleotides, and with rEON2 that is 7 nucleotides shorter than rEON1 at the 3′ end. Combining mEON1 and rEON2 results in a partial HEON (pHEON). Both rEON1 and rEON2 were also produced in a version in which tocopherol according to the formula:

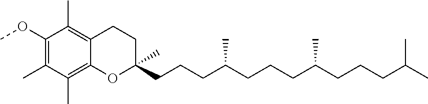

was bound to the 5′ terminus of each sense oligonucleotide through a metabolically cleavable PO linkage. These second nucleic acid strands are given as Toc-rEON1 and Toc-rEON2. Besides that, rEON2 was also produced in a version in which cholesterol according to the formula:

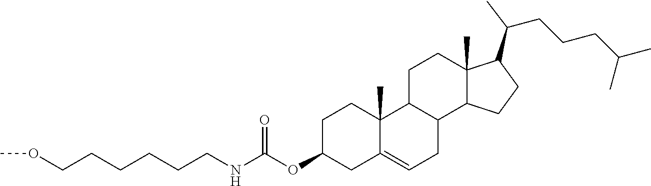

was bound to the 5′ terminus of rEON2 via a C6N linker (depicted) through a PO linkage. This second nucleic acid strand is given as Chol-rEON2. All manufacturing of oligonucleotides, including all modifications, linkages and attachments were according to general procedures known to the person skilled in the art.

For the generation of HEONs, equimolar concentrations of first nucleic acid strands and second nucleic acid strands were mixed in PBS, briefly heated to 95° C., and allowed to cool down to RT. 1 nmol of mEON1 or equimolar (p) HEON complexes were administered by 1 μL intravitreal (IVT) injection to six C57BL/6J mice, in each eye. After 14 days, mice were sacrificed, retinas were isolated and examined for target RNA editing levels.

RNA isolation, cDNA synthesis, dPCR and assessment of RNA editing was performed as generally known to the person skilled in the art. Briefly, to this end, snap frozen tissue samples were thawed and disrupted in TRIzol® reagent (Roche) using the MagNA lyzer (Roche). Samples were exposed to two MagNA lyzer runs of 30 sec each (6500 rpm), allowing for a 90 sec cool-off period in between runs. Samples were then incubated for 2 min at RT to allow complete dissociation of nucleoproteins. Subsequently, chloroform was added to the suspension preparing for phase separation. After centrifuging for 15 min at 12,000×g (4° C.), the aqueous phase that contained RNA was used for further processing. The ReliaPrep™ RNA Cell Miniprep System was used to isolate RNA following the manufacturer's protocol. cDNA synthesis was performed using the Maxima Reverse Transcriptase kit (Thermo Scientific) following the manufacturer's protocol and the primers given below. In short, to avoid cDNA synthesis interference due to secondary structures, 500 ng of RNA was first incubated with dNTP mix (10 mM each), random hexamers and oligoDt at 70° C. for 5 min, then slowly cooled to 10° C. in 10° C. per 15 sec declines. Subsequently, reverse transcriptase buffer and enzyme was added, and samples were incubated at 25° C. for 10 min, 50° C. for 30 min, and 80° C. for 5 min (to deactivate the enzyme). For dPCR analysis, cDNA samples were incubated with a mix of primers and probes specific for wild type mApp exon 17 (HEX), mutant mApp exon 17 (FAM), total mApp exon 4-5 (Cy5) and mRps19 (TEX 615). 12 μl of each sample was loaded onto a QIAcuity nano plate 8.5K 96 wells and run on the QIAcuity (Qiagen) which includes sample partition, PCR, and imaging.

Primers and probes (“+” relates to an LNA
nucleotide at the 3′ side)
mApp exon 17-fw primer:
(SEQ ID NO: 8)
5′-CAACATCACCAGGGTGATGAC-3′
mApp exon 17-rv primer:
(SEQ ID NO: 9)
5′-CATCATCGGACTCATGGTGG-3′
Probe, wt:
(SEQ ID NO: 10)
/5HEX/CGTT+GTCAT+A+G+CAACCGT/3IABkFQ/
Probe, mutant:
(SEQ ID NO: 11)
/56-FAM/CGTTGTCAT+G+G+CAACCG/3IABkFQ/
mApp exon 4-5-fw primer:
(SEQ ID NO: 12)
5′-GCGGATGGATGTTTGTGAGA-3′
mApp exon 4-5-rv primer:
(SEQ ID NO: 13)
5′-GCCATAGTCGUGCAAGTTAGTG-3′
Probe:
(SEQ ID NO: 14)
/5Cy5/CAC ACC+G+TCG+C+CAAA/3IAbRQSp/
mRps19-fw primer:
(SEQ ID NO: 15)
5′-CGAGCTGCTTCCACA-3′
mRps19-rv primer:
(SEQ ID NO: 16)
5′-GCCACACTCTTAGAGCC-3′
Probe:
(SEQ ID NO: 17)
/5TexRd-XN/GCACCTGTACCTCCGAGGTG/3BHQ_2/

FIG. 2C shows the results and the percentage RNA editing observed for the target adenosine in the target mApp transcript. When mEON1 was administered as a single-stranded oligonucleotide alone, some editing could be observed, while rEON2 (the sense strand) alone showed no level of editing. Combining mEON1 and rEON1 in a heteroduplex oligonucleotide complex did not give a higher percentage editing than what was observed with mEON1 alone. When mEON1 was either combined with Toc-rEON1 or Toc-rEON2 a minor increase of RNA editing was observed. However, when mEON1 was complexed in a pHEON with Chol-rEON2 a significant increase (from ~2.5% to ~10%) in RNA editing effect was observed. The data of the left and right retina from each mouse was averaged and given as single data points. This shows that the inventors were able to obtain a higher RNA editing efficiency from a first nucleic acid strand (=mEON1; the EON, or the guide oligonucleotide, or the RNA editing oligonucleotide) in vivo in mouse retina, when that first nucleic acid strand was annealed to a second nucleic acid strand to form a (p) HEON and wherein a hydrophobic moiety was bound to the second nucleic acid strand. As shown here, for this setup, in mouse retina, the best results were obtained when cholesterol as the hydrophobic moiety was bound to the second nucleic acid strand.

### Example 2. In Vivo a to I Editing in mApp Target RNA in Mouse Liver Using Heteroduplex RNA Editing Oligonucleotide Complexes (HEONs)

A similar experiment as described in Example 1 was performed, in which the same oligonucleotides and (p) HEONs were tested for RNA editing in an mAPP target RNA molecule in a variety of tissues of mice. The sequences used and oligonucleotides with their modifications are as shown in FIG. 2A and BC57BL/6J mice were injected intravenously (IV) with 35 mg/kg of mEON1 or equimolar (p) HEON complexes by intravenous bolus injection. After 14 days mice were sacrificed. Subsequently, livers, kidneys, lungs, and spleens were isolated and examined for target RNA editing levels. RNA isolation, cDNA production and ddPCR analysis was generally as described above.

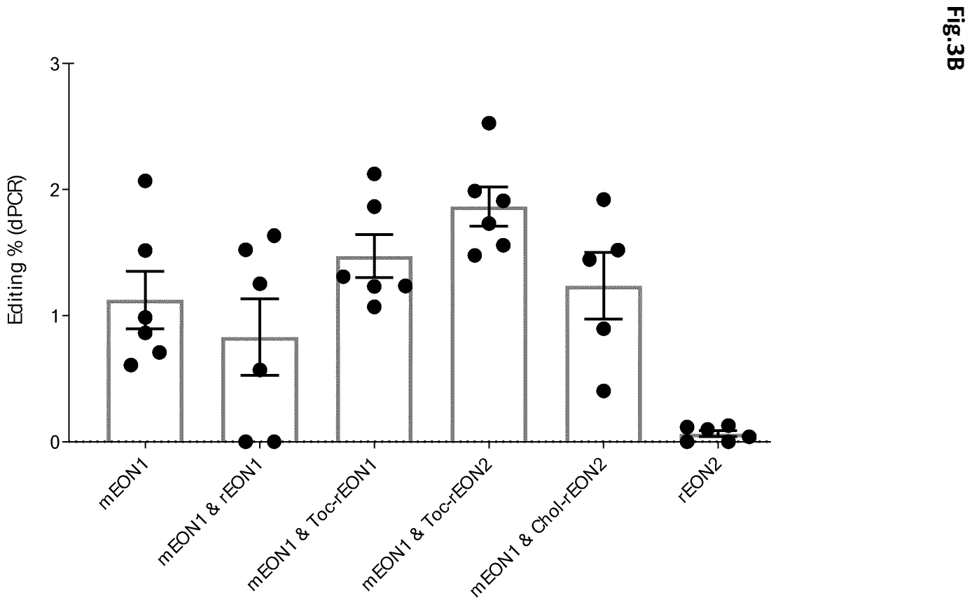

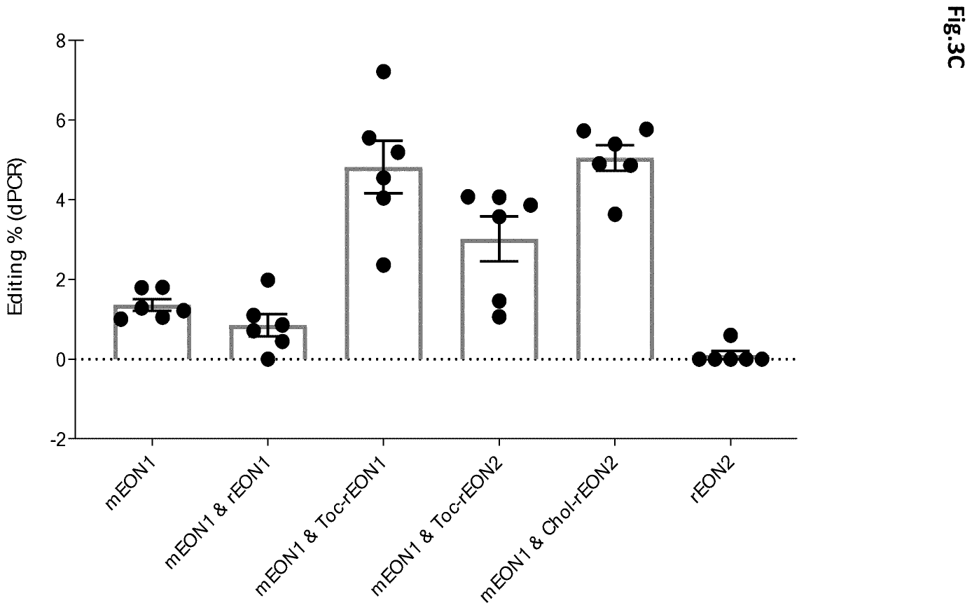

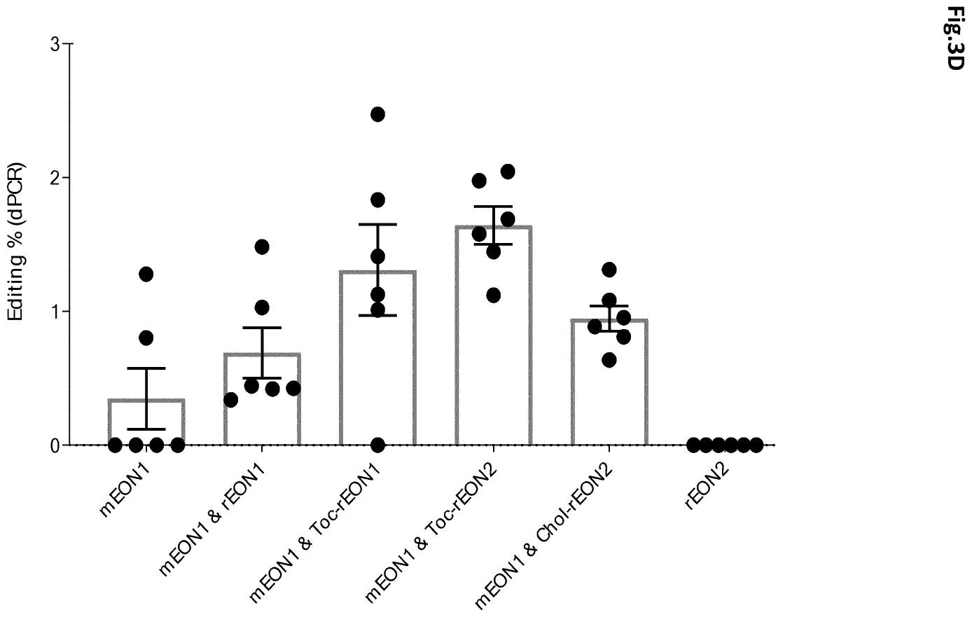

FIG. 3A shows that mEON1 alone produced approximately 2% editing in the liver, and that a HEON complex comprising mEON1 as the first nucleic acid strand and rEON1 as the second nucleic acid strand produced less editing (~0.8%) and that using rEON2 alone did not produce any editing levels. However, when mEON1 was annealed to Toc-rEON1 and Toc-rEON2, significant higher percentages were observed (up to 5%), while combining mEON1 with Chol-rEON2 in a pHEON, editing efficiency was increased somewhat (not significant) but not to the levels as seen with the tocopherol bound rEONs. FIG. 3B shows the results observed in the kidneys. FIG. 3C shows the results observed in the lungs. FIG. 3D shows the results observed in the spleens. FIG. 3E shows the results observed in the heart muscle. In all cases the tocopherol or cholesterol attachments to the second nucleic acid strand gave an increase in RNA editing percentage in comparison to the use of mEON1 alone, indicating that the use of the hydrophobic moiety in a heteroduplex RNA editing oligonucleotide complex is beneficial in providing a more efficient RNA editing effect in vivo. The choice of hydrophobic moiety may depend on the tissue or cells that needs to be targeted.

### Example 3. A to I Editing in mApp Target RNA in Retinal Pigment Epithelial (RPE) Cells after Gymnotic Uptake of Cholesterol Bound HEONs

The efficiency of using EONs as the first nucleic acid strand in a HEON further comprising a second nucleic acid strand bound to cholesterol was further tested with two second nucleic acid strands that were shorter than the first nucleic acid strand. For this, two HEONs were generated: one with RM4238 (see FIG. 2B) as the second nucleic acid strand and one with RM4239 (see FIG. 2B) as the second nucleic acid strand.

5×10⁴ mouse RPE cells were seeded in a 24-well plate on day 0 and treated with 5 μM of oligonucleotide on the same day. Untreated cells were used as a negative control. Five days after adding the oligonucleotides, cells were harvested, and RNA was extracted using the Direct-zol RNA miniprep (Zymo research) kit according to the manufacturer's instructions, and cDNA was prepared using the Maxima reverse transcriptase kit (Thermo Fisher) according to the manufacturer's instructions, with a combination of random hexamer and oligo-dT primers. The cDNA was diluted 10× and 1.1 μL of this dilution was used as template for digital droplet PCR (ddPCR). The ddPCR assay for absolute quantification of nucleic acid target sequences was performed using BioRad's QX-200 Droplet Digital PCR system using general procedures known to the person skilled in the art. 2.2 μL of diluted cDNA obtained from the RT cDNA synthesis reaction was used in a total mixture of 44 μL reaction mixture, including the ddPCR Supermix for Probes no dUTP (Bio Rad), a Taqman SNP genotype assay with the forward and reverse primers mentioned above (exon 17) combined with the gene-specific probes that were also given above.

A total volume of 21 μL PCR mix including cDNA was filled in the middle row of a ddPCR cartridge (BioRad) using a multichannel pipette. The replicates were divided by two cartridges. The bottom rows were filled with 70 μL of droplet generation oil for probes (BioRad). After placing the rubber gasket on the cartridges, droplets were generated in the QX200 droplet generator. 42 μL of oil emulsion from the top row of the cartridge was transferred to a 96-wells PCR plate. The PCR plate was sealed with a tin foil for 4 sec at 170° C. using the PX1 plate sealer, followed by the following PCR program: 1 cycle of enzyme activation for 10 min at 95° C., 40 cycles denaturation for 30 sec at 95° C. and annealing/extension for 1 min at 55.8° C., 1 cycle of enzyme deactivation for 10 min at 98° C., followed by a storage at 8° C. After PCR, the plate was read and analyzed with the QX200 droplet reader.

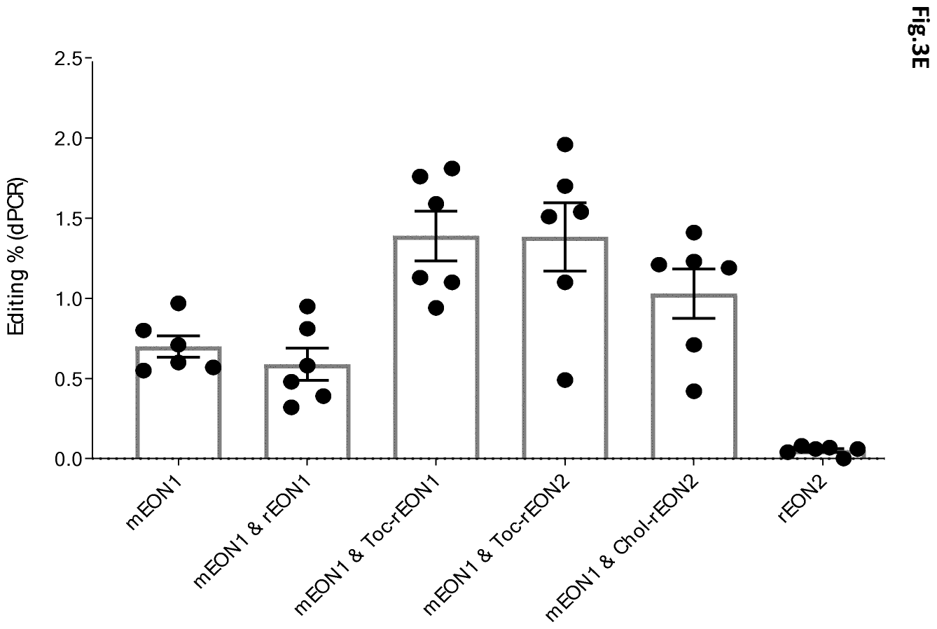

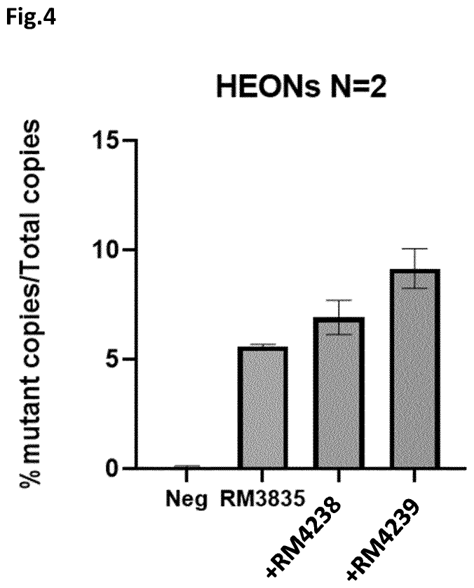

FIG. 4 shows the results of this experiment and clearly indicates that approximately 5% editing can be achieved using mEON1 alone, but that this can be significantly increased by using a partial HEON in which mEON1 acts as the first nucleic acid strand and a cholesterol-bound second nucleic acid strand that is only partially complementary to mEON1 as the second nucleic acid strand.

1. A heteroduplex RNA editing oligonucleotide complex (HEON) for use in the deamination of a target adenosine present in a target RNA molecule in a cell, wherein the HEON comprises a first nucleic acid strand that is annealed to a complementary second nucleic acid strand, wherein the first nucleic acid strand is complementary to a stretch of nucleotides in the target RNA molecule that includes the target adenosine, wherein the first nucleic acid strand when it is hybridized to the target RNA molecule is capable of recruiting an enzyme with deaminase activity present in the cell allowing for the deamination of the target adenosine, and wherein the first and/or second nucleic acid strand is bound to a hydrophobic moiety.

2. The HEON according to claim 1, wherein the hydrophobic moiety is a lipid, a hydrophobic vitamin, or a steroid.

3. The HEON according to claim 2, wherein the hydrophobic vitamin is vitamin E or analog thereof.

4. The HEON according to claim 2, wherein the steroid is cholesterol or analog thereof.

5. The HEON according to any one of claims 1 to 4, wherein the hydrophobic moiety is bound to the 5′ terminus, to the 3′ terminus, and/or to an internal position of the first nucleic acid strand and/or of the second nucleic acid strand.

6. The HEON according to claim 5, wherein the hydrophobic moiety is bound to the 5′ terminus of the second nucleic acid strand.

7. The HEON according to claim 5, wherein the hydrophobic moiety is bound to the 3′ terminus of the second nucleic acid strand.

8. The HEON according to any one of claims 1 to 7, wherein the hydrophobic moiety is bound via a cleavable linker.

9. The HEON according to any one of claims 1 to 8, wherein the HEON is further bound to a ligand that enables cell-specific targeting.

10. The HEON according to claim 9, wherein the ligand comprises a GalNAc moiety.

11. The HEON according to any one of claims 1 to 10, wherein the first nucleic acid strand and/or the second nucleic acid strand comprises one or more nucleosides that comprise a chemical modification that is a mono- or di-substitution at the 2′, 3′ and/or 5′ position of the ribose sugar, selected from the group consisting of:
—OH;
—H;
—F;
substituted or unsubstituted, linear or branched lower (C₁-C₁₀) alkyl, alkenyl, alkynyl, alkaryl, allyl, or aralkyl, that may be interrupted by one or more heteroatoms;
—O—, S—, or N-alkyl;
—O—, S—, or N-alkenyl;
—O—, S—, or N-alkynyl;
—O—, S—, or N-allyl;
—O-alkyl-O-alkyl;
-methoxy;
-aminopropoxy;
-methoxyethoxy;
-dimethylamino oxyethoxy; and
-dimethylaminoethoxyethoxy.

12. The HEON according to any one of claims 1 to 11, wherein the nucleotide in the first nucleic acid strand that is directly opposite the target adenosine is the orphan nucleotide that has the structure of formula I: 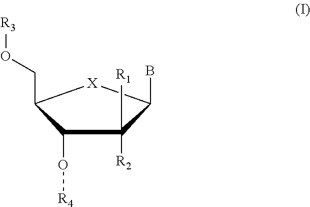
wherein:
X is O, NH, OCH₂, CH₂, Se, or S;
B is a nitrogenous base selected from the group consisting of: cytosine, uracil, isouracil, N3-glycosylated uracil, pseudoisocytosine, 8-oxo-adenine, and 6-amino-5-nitro-2 (1H)-pyridone;
R₁ and R₂ are both selected, independently, from H, OH, F or CH₃;
R₃ is the part of the first nucleic acid strand that is 5′ of the orphan nucleotide, consisting of 7 to 30 nucleotides; and
R₄ is the part of the first nucleic acid strand that is 3′ of the orphan nucleotide, consisting of 4 to 25 nucleotides.

13. The HEON according to any one of claims 1 to 12, wherein the first nucleic acid strand and/or the second nucleic acid strand comprises at least one phosphorothioate (PS), phosphonoacetate, methylphosphonate (MP), phosphoryl guanidine, or PNdmi internucleotide linkage.

14. The HEON according to claim 13, wherein the first nucleic acid strand comprises a single MP linkage at linkage position-1.

15. The HEON according to claim 13 or 14, wherein the first nucleic acid strand comprises a PNdmi linkage at the most terminal linkage at the 5′ and/or 3′ terminus.

16. The HEON according to any one of claims 13 to 15, wherein the first nucleic acid strand and/or the second nucleic acid strand comprises PS linkages linking the most terminal four nucleosides at the 5′ and/or 3′ end.

17. The HEON according to any one of claims 1 to 16, wherein the enzyme with deaminase activity is an endogenous ADAR enzyme, preferably a human ADAR enzyme.

18. The HEON according to any one of claims 1 to 17, wherein the first nucleic acid strand is longer or shorter than the second nucleic acid strand.

19. The HEON according to any one of claims 1 to 18, wherein the first and second nucleic acid strands are fully complementary.

20. A pharmaceutical composition comprising a HEON according to any one of claims 1 to 19, further comprising a pharmaceutically acceptable carrier and/or solvent.

21. The HEON according to any one of claims 1 to 19, or the pharmaceutical composition according to claim 20, for use in the treatment or prevention of a disorder selected from the group consisting of: Hurler Syndrome, alpha-1-antitrypsin (A1AT) deficiency, (familial) hypercholesterolemia, Parkinson's disease, Rett syndrome, Stargardt Disease, Citrullinemia Type 1, autosomal recessive non-syndromic hearing loss, X-linked retinoschisis, argininosuccinate lyase deficiency, Duchenne/Becker muscular dystrophy, Non-Alcoholic Steatohepatitis (NASH), Myotonic dystrophy type I, Myotonic dystrophy type II, Huntington's disease, Usher syndrome (such as Usher syndrome type I, II, and III), Charcot-Marie-Tooth disease, Cystic fibrosis, Alzheimer's disease, albinism, Amyotrophic lateral sclerosis, Asthma, β-thalassemia, Epileptic Encephalopathy, CADASIL syndrome, Chronic Obstructive Pulmonary Disease (COPD), Distal Spinal Muscular Atrophy (DSMA), Dystrophic Epidermolysis bullosa, Epidermolysis bullosa, Fabry disease, Factor V Leiden associated disorders, Familial Adenomatous Polyposis, Galactosemia, Gaucher's Disease, Glucose-6-phosphate dehydrogenase deficiency, Haemophilia, Hereditary Hemochromatosis, Hereditary Cancer predisposing Syndrome, Hunter Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anaemia, Spinal Muscular Atrophy, Tay-Sachs Disease, X-linked immunodeficiency, Sturge-Weber Syndrome, and cancer, such as breast and lung cancer.

22. A method for the deamination of at least one target adenosine in a target RNA in a cell, the method comprising the steps of:
(i) providing the cell with a HEON as defined in any one of claims 1 to 19;
(ii) allowing annealing of the first nucleic acid strand to the target RNA in the cell to form a double stranded nucleic acid molecule;
(iii) allowing an ADAR enzyme to complex with the double stranded nucleic acid molecule between the guide oligonucleotide and the target RNA and to deaminate the target adenosine in the target RNA to an inosine; and
(iv) optionally identifying the presence of the deaminated nucleotide in the target RNA.

23. The method of claim 22, wherein step (iv) comprises:
a) sequencing a region of the target RNA, wherein the region comprises the position of the target adenosine;
b) assessing the presence of a functional, elongated, full length and/or wild type protein when the target adenosine is in a stop codon;
c) assessing, when the target RNA is pre-mRNA, whether splicing of the pre-mRNA was altered by the deamination; or
d) using a functional read-out, wherein the target RNA after the deamination encodes a functional, full length, elongated and/or wild type protein.


SEQUENCE LISTING (17 sequences)

SEQ ID NO: 1 - RNA, 50 nt, Mus musculus
  source 1..50: mol_type = mRNA
tggtgggcggcgttgtcatagcaaccgtgattgtcatcaccctggtgatg

SEQ ID NO: 2 - RNA, 25 nt, synthetic construct
  source 1..25: mol_type = other RNA
  modified_base 12: mod_base = OTHER; note = 6-amino-5-nitro-2(1H)-pyridone
atcacggttgcnatgacaacgccgc

SEQ ID NO: 3 - RNA, 25 nt, synthetic construct
  source 1..25: mol_type = other RNA
gcggcgttgtcatggcaaccgtgat

SEQ ID NO: 4 - RNA, 18 nt, synthetic construct
  source 1..18: mol_type = other RNA
gcggcgttgtcatggcaa

SEQ ID NO: 5 - RNA, 21 nt, synthetic construct
  source 1..21: mol_type = other RNA
gcggcgttgtcatggcaaccg

SEQ ID NO: 6 - RNA, 15 nt, synthetic construct
  source 1..15: mol_type = other RNA
gcggcgttgtcatgg

SEQ ID NO: 7 - RNA, 12 nt, synthetic construct
  source 1..12: mol_type = other RNA
gcggcgttgtca

SEQ ID NO: 8 - DNA, 21 bp, synthetic construct
  source 1..21: mol_type = other DNA
caacatcaccagggtgatgac

SEQ ID NO: 9 - DNA, 20 bp, synthetic construct
  source 1..20: mol_type = other DNA
catcatcggactcatggtgg

SEQ ID NO: 10 - DNA, 18 bp, synthetic construct
  source 1..18: mol_type = other DNA
cgttgtcatagcaaccgt

SEQ ID NO: 11 - DNA, 17 bp, synthetic construct
  source 1..17: mol_type = other DNA
cgttgtcatggcaaccg

SEQ ID NO: 12 - DNA, 20 bp, synthetic construct
  source 1..20: mol_type = other DNA
gcggatggatgtttgtgaga

SEQ ID NO: 13 - DNA, 22 bp, synthetic construct
  source 1..22: mol_type = other DNA
  modified_base 11: mod_base = OTHER; note = Deoxyuridine
gccatagtcgtgcaagttagtg

SEQ ID NO: 14 - DNA, 15 bp, synthetic construct
  source 1..15: mol_type = other DNA
cacaccgtcgccaaa

SEQ ID NO: 15 - DNA, 15 bp, synthetic construct
  source 1..15: mol_type = other DNA
cgagctgcttccaca

SEQ ID NO: 16 - DNA, 17 bp, synthetic construct
  source 1..17: mol_type = other DNA
gccacactcttagagcc

SEQ ID NO: 17 - DNA, 20 bp, synthetic construct
  source 1..20: mol_type = other DNA
gcacctgtacctccgaggtg